(19) United States
(12) Patent Application Publication
(10) Pub. No.: US 2020/0150116 A1
(43) Pub. Date: May 14, 2020

(54) SINGLE-STEP ATPS ENHANCED LFA DIAGNOSTIC DESIGN

(71) Applicant: The Regents of the University of California, Oakland, CA (US)

(72) Inventors: Daniel Takashi Kamei, Monterey Park, CA (US); Benjamin Ming Wu, San Marino, CA (US); Garrett L. Mosley, Newport Beach, CA (US); Yin To Chiu, Irvine, CA (US); David Yuan Pereira, Los Angeles, CA (US); Chloe Michelle Wu, San Marino, CA (US); Yue Han, Plano, TX (US); So Youn Lee, Cupertino, CA (US)

(21) Appl. No.: 16/616,923

(22) PCT Filed: May 30, 2018
(86) PCT No.: PCT/US18/35204

§ 371 Date: Nov. 25, 2019

Related U.S. Application Data
(60) Provisional application No. 62/513,347, filed on May 31, 2017.

(51) Int. Cl. G01N 33/558; G01N 33/569

(52) U.S. Cl. CPC G01N 33/558; G01N 33/56927

(57) ABSTRACT

In various embodiments single-step ATPS paper-based diagnostic assays are provided that exploit the concept of sequential resolubilization of ATPS components to give rise to the desired phase separation behavior within paper. In one illustrative embodiment, a wick is provided for concentrating an analyte within an aqueous two-phase extraction system in a paper, where the wick comprises a paper configured to receive a sample where the paper comprises a first region containing a first component of an aqueous two-phase system (ATPS) where the first component is in a dry form, and a second region containing a second component of an aqueous two-phase system (ATPS) where the second component is in a dry form; and where said first region and the second region are disposed so that when said wick is contacted with a fluid sample, the first component of said ATPS is hydrated before the second component. In certain embodiments the first and second component are disposed so they are hydrated substantially simultaneously.

### CROSS-REFERENCE TO RELATED APPLICATIONS

This application claims benefit of and priority to U.S. Ser. No. 62/513,347, filed on May 31, 2017, which is incorporated herein by reference in its entirety for all purposes.

### STATEMENT OF GOVERNMENTAL SUPPORT

This invention was made with Government support under Grant Number 1549003, awarded by the National Science Foundation. The Government has certain rights in the invention.

### BACKGROUND

Infectious diseases such as chlamydia and HIV greatly affect both developed and developing countries. Chlamydia is a sexually transmitted infection (STI) caused by the bacterium Chlamydia trachomatis which, if left untreated, can lead to pelvic inflammatory disease in women and cause permanent damage to the reproductive system (Hafner (2015) Contraception, 92: 108-115). The prevalence of chlamydia has been steadily rising in the United States since 1993, with over 1.4 million new chlamydia infections reported in 2014 (Centers for Disease Control and Prevention (2014) Sexually Transmitted Disease Surveillance 2014: 1-176). Although chlamydia is relatively straightforward to treat, and shows no signs of emerging resistance to primary pharmacological treatment options (Krupp & Madhivanan (2015) Indian J Sex Transm Dis 36: 3-8), it is still one of the most common STIs in the United States (Centers for Disease Control and Prevention (2014) Sexually Transmitted Disease Surveillance 2014: 1-176). HIV, on the other hand, is caused by the human immunodeficiency virus which attacks the body's immune system, specifically the CD4 cells. In 2015 alone, there were about 2.1 million new cases of HIV worldwide, and about 39,513 people were diagnosed with HIV in the United States (CDC (2015) HIV Surveill. Rep. 27: 1-82). One approach for addressing the increasing prevalence of chlamydia and HIV is through low-cost point-of-care (POC) screening of at-risk populations, which has shown promising results in theoretical models (Huang et al. (2013) Sex Transm. Infect. 89: 108-114. doi: 10.1136/sextrans-2011-050355; Miller (1998) Sex. Transm. Infect. 25: 201-211) and isolated trial studies (Mahilum-Tapay et al. (2007) BMJ, 335: 1190-1194; Low et al (2006) Lancet, 368: 2001-2016).

Unfortunately, current gold standard laboratory-based diagnostics, such as ELISA tests, nucleic acid amplification tests (NAATs), or cell culture methods, are not suitable for POC screening. This is due to the high cost of equipment, the requirement for trained personnel, and the lengthy time to result. In contrast, paper-based diagnostics are a more suitable technology, with two components that are necessary for effective large scale screening: on-site diagnosis and treatment within the same visit, and administration by untrained or minimally trained personnel. The most commonly used paper diagnostic is the lateral-flow immunoassay (LFA), a visually interpreted antibody-based diagnostic recognized for its widespread use in pregnancy tests (Wong & Tse (2009) Lateral Flow Immunoassay, 1st ed. Springer, New York). Unfortunately, chlamydia LFA tests are currently not sensitive enough to be effective diagnostics (Land et al. (2009) Hum. Reprod. Update, 16: 189-204), a limitation that most paper-based diagnostics for infectious diseases suffer from (Gubala et al. (2012) Anal. Chem. 84: 487-515). Although HIV LFA tests are more established in the consumer market than chlamydia LFA tests, there is still room for their sensitivity to be improved to further minimize the risk of false negatives and potential transmission of the virus.

Significant efforts have been made in recent years to improve the sensitivity of paper-based assays. Some key innovations include work with two-dimensional paper networks by the Yager lab (Fu et al. (2010) Sensors Actuators, B Chem. 149: 325-328; Fu et al. (2010) Lab Chip, 10: 918-920; Osborn et al. (2010) Lab Chip, 10: 2659-2565; Fu et al (2011) Microfluid Nanofluidics, 10: 29-35; Kauffman et al. (2010) Lab Chip, 10: 2614-2617; Fridley et al. (2012) Lab Chip, 12: 4321; Fu et al. (2012) Anal. Chem. 84: 4574-4579; Lutz et al. (2013) Lab Chip, 13: 2840-2847) and microfluidic paper-based analytical devices by the Whitesides lab (Mosadegh et al. (2015) Biomaterials, 52: 262-271; Thuo et al. (2014) Chem. Mater. 26: 4230-4237; Lan et al. (2014) Anal. Chem. 86: 9548-9553; Badu-Tawiah et al. (2014) Lab Chip, 15: 655-659). Previously, our lab developed an equipment-free method to thermodynamically pre-concentrate target analytes prior to their application to LFA tests. In short, this is accomplished by utilizing aqueous two-phase systems (ATPSs), which separate into two distinct liquid phases, where the target analyte partitions extremely into one of those phases, effectively concentrating the target. In the first approach, our 3-step diagnostic process involved (i) mixing a large volume of target solution with ATPS components, (ii) waiting for macroscopic phase separation, and (iii) extracting and applying the concentrated target phase to the LFA test. With this method, we demonstrated an improvement in the limit of detection for both large viruses (Jue et al. (2014) Biotechnol. Bioeng. 111: 2499-2507; Mashayekhi et al. (2010) Anal. Bioanal. Chem. 398: 2955-2961) and small protein targets (Mashayekhi et al. (2012) Anal. Bioanal. Chem. 404: 2057-2066; Chiu et al. (2014) Ann. Biomed. Eng. 42(11): 2322-2332). Recently, we discovered that the phase separation process is expedited when the ATPS flows through paper, reducing the overall diagnostic time from hours down to minutes by eliminating the waiting and extraction steps. Using this phenomenon, our lab demonstrated the ability to simultaneously concentrate and detect protein biomarkers within paper (Chiu et al. (2014) Lab chip, 14: 3021-3028; Pereira et al. (2015) Anal. Chim. Acta. 882: 83-89). This diagnostic process still required an initial ATPS component mixing step prior to application of the solution to an LFA strip, which can be suitable for applications that already require initial mixing into a predetermined buffer (e.g., a swab-based diagnostic).

### SUMMARY

In various embodiments described herein are single-step ATPS paper-based diagnostic assays based on the novel concept of sequential resolubilization of ATPS components to give rise to the desired phase separation behavior within paper.

Various embodiments contemplated herein may include, but need not be limited to, one or more of the following:

### Embodiment 1

A wick for concentrating an analyte within an aqueous two-phase extraction system in a paper, said wick comprising:

a paper configured to receive a sample wherein said paper comprises:
a first region containing a first component of an aqueous two-phase system (ATPS) where said first component is in a dry form; and
a second region containing a second component of an aqueous two-phase system (ATPS) where said second component is in a dry form;
wherein said first region and said second region are disposed so that when said wick is contacted with a fluid sample, said first component of said ATPS is hydrated before said second component; or wherein said paper comprises:

a region containing both a first component of an aqueous two-phase system (ATPS) and a second component of an aqueous two-phase system where said first component and said second component are in a dry form so that when said wick is contacted with a fluid sample, said first component of said ATPS and said second component of said ATPS are hydrated at substantially the same time.

### Embodiment 2

The wick of embodiment 1, wherein said paper comprises:

a first region containing a first component of an aqueous two-phase system (ATPS) where said first component is in a dry form; and

a second region containing a second component of an aqueous two-phase system (ATPS) where said second component is in a dry form; and

wherein said first region and said second region are disposed so that when said wick is contacted with a fluid sample, said first component of said ATPS is hydrated before said second component.

### Embodiment 3

The wick according to any one of embodiments 1-2, wherein said wick is configured so that the first component of said ATPS when hydrated flows into said second component of said ATPS hydrating said second component to provide a mixed phase that separates into a first phase comprising said first component and a second phase comprising said second component as the ATPS moves through said wick.

### Embodiment 4

The wick according to any one of embodiments 1-3, wherein said first component and said second component are components of a polymer/salt ATPS where said first component comprises a salt and said second component comprises a polymer.

### Embodiment 5

The wick of embodiment 4, wherein said salt comprise one or more salts selected from the group consisting of potassium phosphate, sodium sulfate, magnesium sulfate, ammonium sulfate, sodium citrate, magnesium chloride, magnesium citrate, magnesium phosphate, sodium chloride, potassium citrate, and potassium carbonate.

### Embodiment 6

The wick of embodiment 5, wherein said salt comprises potassium phosphate.

### Embodiment 7

The wick according to any one of embodiments 4-6, wherein said salt ranges from about 0.1% w/w to about 40% w/w, or from about 1% w/w up to about 30% w/w, or from about 5% w/w up to about 25% w/w, or from about 10% w/w up to about 20% w/w.

### Embodiment 8

The wick of embodiment 7, wherein said salt is present at about 15% (w/w).

### Embodiment 9

The wick according to any one of embodiments 4-8, wherein said polymer comprises a polymer selected from the group consisting of polyethylene glycol (PEG), ethylene/propylene copolymer (e.g., UCON™ 50-HB), propylene glycol (PPG), methoxypolyethylene glycol, and polyvinyl pyrrolidone.

### Embodiment 10

The wick of embodiment 9, wherein said polymer comprises polyethylene glycol (PEG).

### Embodiment 11

The wick of embodiment 10, wherein said PEG has a molecular weight that ranges from about 1,000 to about 100,000, or from about 4,000 to about 50,000, or from about 5,000 up to about 40,000, or up to about 30,000, or up to about 20,000.

### Embodiment 12

The wick of embodiment 11, wherein said polymer comprises polyethylene glycol (PEG) 8000 MW.

### Embodiment 13

The wick according to any one of embodiments 4-12, wherein said polymer comprises about 1% w/w to about 30% w/w, or from about 5% w/w up to about 25% w/w, or from about 10% w/w up to about 25% w/w, or from about 10% w/w up to about 20% w/w polymer.

### Embodiment 14

The wick of embodiment 13, wherein said polymer comprises about 10% (w/w).

### Embodiment 15

The wick according to any one of embodiments 1-14, wherein said paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), and combinations thereof.

### Embodiment 16

The wick of embodiment 15, wherein said paper comprises fiberglass.

### Embodiment 17

The wick according to any one of embodiments 1-16, wherein said wick comprises a plurality of layers of said paper.

### Embodiment 18

The wick of embodiment 17, wherein said wick comprises at least 3, or at least 4, or at least 5, or at least 6, or at least 7, or at least 8, or at least 9, or at least 10, or at least 15, or at least 20 layers of said paper.

### Embodiment 19

The wick of embodiment 17, wherein said wick comprises about 5 layers of said paper.

### Embodiment 20

The wick according to any one of embodiments 1-19, wherein an ATPS component free region is disposed between said first region and said second region.

### Embodiment 21

The wick according to any one of embodiments 1-19, wherein said first region is disposed adjacent to said second region.

### Embodiment 22

The wick according to any one of embodiments 1-21, wherein said wick comprises a sample application region.

### Embodiment 23

The wick of embodiment 22, wherein said sample application region comprises a sample pad.

### Embodiment 24

The wick according to any one of embodiments 1-23, wherein said wick tapers in a region downstream from said second region and upstream of a lateral flow assay (LFA) when an LFA is in fluid communication with said wick.

### Embodiment 25

The wick according to any one of embodiments 1-24, wherein said wick is configured to be coupled to a lateral flow immunoassay (LFA) and provide fluid communication from said wick to said LFA.

### Embodiment 26

The wick of embodiment 25, wherein said wick is configured to be coupled to an LFA so that plane of wick is perpendicular to the plane of the LFA.

### Embodiment 27

The wick of embodiment 25, wherein said wick is configured to be coupled to an LFA so that plane of wick is parallel to the plane of the LFA.

### Embodiment 28

The wick of embodiment 25, wherein said wick is coupled to a lateral flow immunoassay.

### Embodiment 29

The wick of embodiment 28, wherein said wick is coupled to an LFA so that plane of said wick is parallel to the plane of the LFA.

### Embodiment 30

The wick of embodiment 28, wherein said wick is coupled to an LFA so that plane of said wick is perpendicular to the plane of the LFA.

### Embodiment 31

The wick according to any one of embodiments 28-30, wherein said lateral flow assay comprises:

an LFA paper comprising:
a conjugate region containing a conjugate comprising an indicator moiety attached to a binding moiety that binds to the analyte to be detected, or configured to receive a nanoconjugate complexed with said analyte;
an absorbent region; and
a detection zone comprising a moiety that captures an analyte/nanoconjugate complex.

### Embodiment 32

The wick of embodiment 31, wherein said detection zone comprise a detection line.

### Embodiment 33

The wick according to any one of embodiments 31-32, wherein said LFA comprises a control zone comprising a moiety that captures an analyte/nanoconjugate complex and said nanoconjugate absent of said analyte.

### Embodiment 34

The wick according to any one of embodiments 31-33, wherein said control zone comprises a control line.

### Embodiment 35

The wick according to any one of embodiments 31-34, wherein said conjugate region comprises a conjugate pad.

### Embodiment 36

The wick according to any one of embodiments 31-35, wherein said absorbent region comprises an absorbent pad.

### Embodiment 37

The wick according to any one of embodiments 31-36, wherein said LFA paper is the same material as the paper comprising said wick.

### Embodiment 38

The wick according to any one of embodiments 31-37, wherein said LFA paper is a different material than the paper comprising said wick.

### Embodiment 39

The wick according to any one of embodiments 31-38, wherein said LFA paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

### Embodiment 40

The wick of embodiment 39, wherein said LFA paper comprises nitrocellulose.

### Embodiment 41

The wick of embodiment 39, wherein said LFA paper comprises fiberglass.

### Embodiment 42

The wick according to any one of embodiments 22-23 or 31-41, wherein the sample application region of said wick or the conjugate region of said LFA contains a nanoconjugate comprising an indicator moiety attached to an analyte binding moiety that binds to the analyte to be detected.

### Embodiment 43

The wick of embodiment 42, wherein said analyte binding moiety is selected from the group consisting of an antibody, a lectin, a protein, a glycoprotein, a nucleic acid, monomeric nucleic acid, a polymeric nucleic acid, an aptamer, an aptazyme, a small molecule, a polymer, a lectin, a carbohydrate, a polysaccharide, a sugar, and a lipid.

### Embodiment 44

The wick of embodiment 43, wherein said analyte binding moiety comprises an antibody that binds to said analyte.

### Embodiment 45

The wick according to any one of embodiments 42-44, wherein said indicator comprises a moiety selected from the group consisting of a colorimetric indicator, a fluorescent indicator, and a moiety that can be bound by a construct comprising a colorimetric or fluorescent indicator.

### Embodiment 46

The wick according to any one of embodiments 42-45, wherein said indicator comprise a material selected from the group consisting of a synthetic polymer, a metal, a mineral, a glass, a quartz, a ceramic, a biological polymer, a plastic, and combinations thereof.

### Embodiment 47

The wick according to any one of embodiments 42-46, wherein said indicator comprises a colorimetric indicator.

### Embodiment 48

The wick of embodiment 47, wherein said indicator comprises a gold nanoparticle.

### Embodiment 49

A system for the detection of an analyte, said system comprising:

a container containing a dried nanoconjugate comprising an indicator moiety attached to an analyte binding moiety that binds to said analyte; and

a device comprising a first paper containing components of an aqueous two-phase system where said first paper is in fluid communication with a lateral flow assay (LFA), and where said first paper comprises:
a first region containing a first component of an aqueous two-phase system (ATPS) where said first component is in a dry form; and
a second region containing a second component of an aqueous two-phase system (ATPS) where said second component is in a dry form; wherein:
said first region and said second region are disposed so that when said wick is contacted with a fluid sample, said first component of said ATPS is hydrated before said second component; or
said first region and said second region are the same region and said first component and second component are each distributed over substantially the same region.

### Embodiment 50

The system of embodiment 49, wherein said first region and said second region are the same region and said first component and second component are each distributed over substantially the same region.

### Embodiment 51

The system according to any one of embodiments 49-50, wherein said first component and said second component are components of a polymer/salt ATPS where said first component comprises a salt and said second component comprises a polymer.

### Embodiment 52

The system of embodiment 51, wherein said salt comprise one or more salts selected from the group consisting of potassium phosphate, sodium sulfate, magnesium sulfate, ammonium sulfate, sodium citrate, magnesium chloride, magnesium citrate, magnesium phosphate, sodium chloride, potassium citrate, and potassium carbonate.

### Embodiment 53

The system of embodiment 52, wherein said salt comprises potassium phosphate.

### Embodiment 54

The system according to any one of embodiments 51-53, wherein said polymer comprises a polymer selected from the group consisting of polyethylene glycol (PEG), ethylene/propylene copolymer (e.g., UCON™ propylene glycol (PPG), methoxypolyethylene glycol, and polyvinyl pyrrolidone.

### Embodiment 55

The system of embodiment 54, wherein said polymer comprises ethylene/propylene copolymer (e.g., UCON™ 50-HB).

### Embodiment 56

The system according to any one of embodiments 49-55, wherein said first paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

### Embodiment 57

The system of embodiment 56, wherein said first paper comprises fiberglass.

### Embodiment 58

The system according to any one of embodiments 49-57, wherein said first paper comprises a single layer of said paper.

### Embodiment 59

The system according to any one of embodiments 49-57, wherein said first paper comprises a plurality of layers of said paper.

### Embodiment 60

The system of embodiment 59, wherein said first paper comprises at least 3, or at least 4, or at least 5, or at least 6, or at least 7, or at least 8, or at least 9, or at least 10, or at least 15, or at least 20 layers of said paper.

### Embodiment 61

The system according to any one of embodiments 49-60, wherein a spacer is disposed between said first paper and said lateral flow assay where said spacer provides fluid communication between said first paper and said lateral flow assay.

### Embodiment 62

The system of embodiment 61, wherein said spacer is treated to reduce non-specific binding of analyte and/or nanoconjugate and/or nanoconjugate/analyte complex.

### Embodiment 63

The system of embodiment 62, wherein said spacer is treated with BSA.

### Embodiment 64

The system according to any one of embodiments 62-63, wherein said spacer comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

### Embodiment 65

The system of embodiment 64, wherein said spacer paper comprises fiberglass.

### Embodiment 66

The system according to any one of embodiments 49-60, wherein said paper is disposed adjacent to lateral flow assay.

### Embodiment 67

The system according to any one of embodiments 49-66, wherein said lateral flow assay comprises:

an LFA paper comprising:
an absorbent region; and
a detection zone comprising a moiety that captures an analyte/nanoconjugate complex.

### Embodiment 68

The system of embodiment 67, wherein said detection zone comprises a detection line.

### Embodiment 69

The system according to any one of embodiments 67-68, wherein said LFA comprises a control zone comprising a moiety that captures an analyte/nanoconjugate complex and said nanoconjugate absent the presence of said analyte.

### Embodiment 70

The system of embodiment 69, wherein said control zone comprises a control line.

### Embodiment 71

The system according to any one of embodiments 67-70, wherein said absorbent region comprises an absorbent pad.

### Embodiment 72

The system according to any one of embodiments 67-71, wherein said LFA paper is the same material as said first paper.

### Embodiment 73

The system according to any one of embodiments 67-71, wherein said LFA paper is a different material than said first paper.

### Embodiment 74

The system according to any one of embodiments 67-73, wherein said LFA paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

### Embodiment 75

The system of embodiment 74, wherein said LFA paper comprises nitrocellulose.

### Embodiment 76

The system according to any one of embodiments 49-75, wherein analyte binding moiety is selected from the group consisting of an antibody, a lectin, a protein, a glycoprotein, a nucleic acid, monomeric nucleic acid, a polymeric nucleic acid, an aptamer, an aptazyme, a small molecule, a polymer, a lectin, a carbohydrate, a polysaccharide, a sugar, and a lipid.

### Embodiment 77

The system of embodiment 76, wherein said analyte binding moiety comprises an antibody that binds to said analyte.

### Embodiment 78

The system according to any one of embodiments 76-77, wherein said indicator comprises a moiety selected from the group consisting of a colorimetric indicator, a fluorescent indicator, and a moiety that can be bound by a construct comprising a colorimetric or fluorescent indicator.

### Embodiment 79

The system according to any one of embodiments 76-78, wherein said indicator comprise a material selected from the group consisting of a synthetic polymer, a metal, a mineral, a glass, a quartz, a ceramic, a biological polymer, a plastic, and combinations thereof.

### Embodiment 80

The system according to any one of embodiments 76-79, wherein said indicator comprises a colorimetric indicator.

### Embodiment 81

The system of embodiment 80, wherein said indicator comprises a gold nanoparticle.

### Embodiment 82

A method of detecting and/or quantifying an analyte in a sample, said method comprising:

providing an aqueous solution or suspension comprising said sample; and

applying said solution to a wick according to any one of embodiments 1-48 where said solution sequentially hydrates said first component and said second component as said solution migrates through said wick and partitions said analyte into a phase of said ATPS;

delivering said ATPS into said lateral flow assay; and

detecting and/or quantifying said analyte in said lateral flow assay if said analyte is present.

### Embodiment 83

The method of embodiment 82, wherein said delivering comprises contacting a wick according to any one of embodiments 1-30 with a sample receiving region of said lateral flow assay.

### Embodiment 84

The method of embodiment 82, wherein said wick is in fluid communication with a said wick and said ATPS flows into said LFA.

### Embodiment 85

The method of embodiment 84, wherein said wick is a wick according to any one of embodiments 28-48.

### Embodiment 86

A method of detecting and/or quantifying an analyte in a sample, said method comprising:

providing a system according to any one of embodiments 49-81;

introducing said sample into said container containing a dried nanoconjugate to hydrate said nanoconjugate and to contact said nanoconjugate with said sample where said nanoconjugate forms a nanoconjugate/analyte complex when said analyte is present in said sample;

contacting the region of said device comprising said components of an aqueous two-phase system and hydrating said components where said hydrated components flow through said lateral flow assay; and

detecting and/or quantifying said analyte in said lateral flow assay if said analyte is present.

### Embodiment 87

The method according to any one of embodiments 82-86, wherein said sample is not processed prior to application to said device.

### Embodiment 88

The method according to any one of embodiments 82-86, wherein said sample is diluted prior to application to said device.

### Embodiment 89

The method of embodiment 88, wherein said sample is diluted with phosphate-buffered saline (PBS).

### Embodiment 90

The method according to any one of embodiments 82-89, wherein said subject is a human.

### Embodiment 91

The method according to any one of embodiments 82-89, wherein said subject is a non-human mammal.

### Embodiment 92

The method according to any one of embodiments 82-91, wherein said sample is selected from the group consisting of a biological sample (e.g., oral fluid or tissue sample, nasal fluid, urine, blood or blood fraction, cerebrospinal fluid, lymph, tissue biopsies, vaginal samples, and the like), a food sample, and an environmental sample.

### Embodiment 93

The method according to any one of embodiments 82-92, wherein said analyte comprises a bacterium, a fungus, a protozoan, a virus, or a component thereof.

### Embodiment 94

The method according to any one of embodiments 82-92, wherein said analyte comprises a marker of an infection.

### Embodiment 95

The method of embodiment 94, wherein said marker comprises an antibody directed against the infecting pathogen (e.g., an anti-HIV antibody).

### Embodiment 96

A kit comprising:

a container containing a wick according to any one of embodiments 1-48;

and/or

a container containing the container and/or the device of the system according to any one of embodiments 49-82.

### Definitions

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double-stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments. In addition, it is possible that nucleic acids of the present invention can alternatively be triple-stranded.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example, Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331).

An aptamer is an antibody-analogue formed from nucleic acids. An aptazyme is an enzyme analogue, formed from nucleic acids. In particular, an aptazyme can function to change configuration to capture a specific molecule, only in the presence of a second, specific, analyte. Aptamers may not even require the binding of the first label to be detected in some assays, such as nano-CHEM-FET, where the reconfiguration would be detected directly.

The term “binding moiety”, or a member of a “binding pair” refers to molecules that specifically bind other molecules, cells, microorganisms, and the like to form a binding complex such as antibody-antigen, lectin-carbohydrate, nucleic acid-nucleic acid, biotin-avidin, etc. Such binding moieties include, but are not limited to, monomeric or polymeric nucleic acids, aptamers, aptazymes, proteins, polysaccharides, sugars, lectins, and the like (see, e.g., Haugland, “Handbook of Fluorescent Probes and Research Chemicals” (Sixth Edition)), and any of the molecules capable of forming a binding pair as described above.

The phrase “specifically binds” indicates that the molecule binds preferentially to the target of interest or binds with greater affinity to the target (analyte) than to other molecules. For example, an antibody will selectively bind to the antigen against which it was raised. A DNA molecule will bind to a substantially complementary sequence and not to unrelated sequences under stringent conditions. Specific binding can refer to a binding reaction that is determinative of the presence of a target in a heterogeneous population of molecules (e.g., proteins and other biologics). Thus, under designated conditions (e.g. immunoassay conditions in the case of an antibody or stringent hybridization conditions in the case of a nucleic acid), the specific ligand or antibody binds to its particular “target” molecule and does not bind in a significant amount to other molecules present in the sample.

The term small organic molecules refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term analyte refers to any moiety that is to be detected. Analytes include, but are not limited to particular biomolecules (proteins, antibodies, nucleic acids), bacteria or components thereof, viruses or components thereof (e.g., coat proteins), fungi or components thereof, protozoa or components thereof, drugs, toxins, food pathogens, and the like.

The term “paper”, as used herein, is not limited to thin sheets from the pulp of wood or other fibrous plant substances although, in certain embodiments the use of such papers in the devices described herein is contemplated. Papers more generally refer to porous materials often in sheet form, but not limited thereto that allow a fluid to flow through.

### BRIEF DESCRIPTION OF THE DRAWINGS

FIG. 1 shows a schematic of a typical lateral-flow immunoassay test strip (top) and the sandwich format of a lateral-flow immunoassay (bottom).

FIG. 2 illustrates the PEG/salt ATPS component rehydration order. Time-lapse visualization of phase separation within a single sheet of the ARROW design when the PEG and potassium phosphate were rehydrated in separate regions, and when they were rehydrated as a mixture. Close up images are shown of the downstream region where phase separation occurred, and therefore, the first image is at t=6 s instead of t=0. Visualization and identification of the PEG-rich phase, PEG-poor phase, and macroscopically mixed domain regions were accomplished by flowing a suspension of BSA-DGNPs and Brilliant Blue dye.

FIG. 3 illustrates the UCON/salt ATPS component rehydration order. Time-lapse visualization of phase separation within a single fiberglass strip when the UCON-50-HB-5100 and potassium phosphate were rehydrated in separate regions, and when they were rehydrated as a mixture. Images were cropped to contain the same area of a strip in order to observe relative flow rates. Visualization and identification of the UCON-rich phase, UCON-poor phase, and macroscopically mixed domain regions were accomplished by flowing a suspension of BSA-GNPs and Brilliant Blue dye.

FIG. 4, panels a-b, illustrates the dynamics of phase separation. Panel a) Time-lapse images were taken of the ARROW with separated two-phase components during the process of fluid flow. The fluid consisted of a suspension of BSA-DGNPs and Brilliant Blue dye, which allowed for visualization of the phase separation. Panel b) Time-lapse images were taken of the mixed UCON/salt design during the process of rehydration by a suspension of BSA-GNPs and Brilliant Blue dye.

FIGS. 5A and 5B shows one illustrative embodiments of an integrated ARROW and LFA diagnostic design layout. FIG. 5A shows integrated ARROW and LFA diagnostic (note, in certain embodiments, fiberglass can be replaced with other materials).

FIG. 5B an integrated ARROW and LFA diagnostic design layout and includes a photo of the ARROW and SEM images of the dehydrated PEG on fiberglass, blank fiberglass, and dehydrated potassium phosphate on fiberglass. In the illustrated embodiment, the top and bottom tips of the fiberglass paper sheet were also blank fiberglass.

FIG. 6 illustrates one embodiment of an integrated TUBE and LFA design, which includes the sample tube containing the dried GNP conjugates and the test strip containing the UCON/salt ATPS dehydrated into a fiberglass pad. SEM images of the UCON/salt pad, the BSA-treated spacer, and the nitrocellulose membrane are also shown.

FIG. 7 illustrates improvement in the limit of detection of C. trachomatis LFA by incorporation of the ARROW. Comparison of LFA results at varying C. trachomatis concentrations, with and without the ARROW is presented. Test lines are located on the bottom of the LFA strips and control lines are located on the top of the LFA test strips. Negative control results are shown in the leftmost panels for 0 ng μL⁻¹ C. trachomatis.

FIG. 8 illustrates the improvement in the limit of detection of human IgM LFA by incorporation of the TUBE. A comparison of LFA results at varying human IgM concentrations, with and without the TUBE is presented. Test lines are located on the bottom of the LFA strips and control lines are located on the top of the LFA test strips. Negative control results are shown in the leftmost panels.

FIG. 9, panels a-b, shows plots of the quantified LFA test line intensities for the ARROW/LFA system and the LFA only system (panel a), and the TUBE/LFA system and the LFA only system (panel b).

### DETAILED DESCRIPTION

Numerous diagnostic applications can benefit from the direct addition of a sample without additional mixing with other solutions and buffers. In various embodiments described herein are single-step ATPS paper-based diagnostic assays based on the novel concept of sequential resolubilization of ATPS components to give rise to the desired phase separation behavior within paper. As a proof of principle, this concept was demonstrated using two different polymer/salt ATPSs in two different diagnostic applications—one to detect C. trachomatis for a chlamydia diagnostic, and the other to detect human immunoglobulin M (IgM) in a potential HIV antibody diagnostic application.

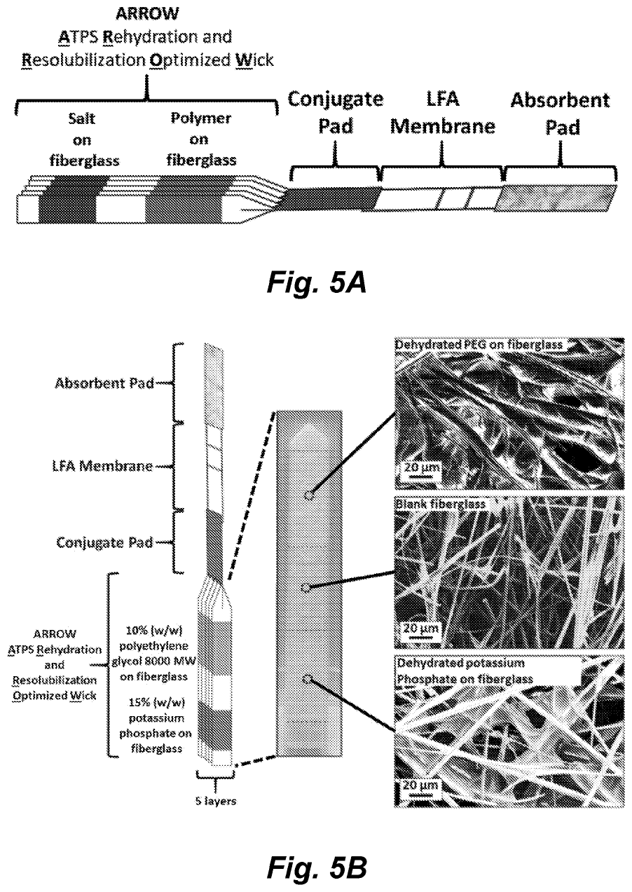

The chlamydia diagnostic utilized an ATPS rehydration and resolubilization optimized wick (designated as the ARROW) that, in the illustrated embodiment, employed a polyethylene glycol and potassium phosphate (PEG/salt) ATPS. In this design, one embodiment of which is illustrated in FIGS. 5A and 5B, the sample solution is added to the device, and the solution directly resolubilizes the ATPS components during flow, resulting in phase separation and subsequent concentration of C. trachomatis within paper.

The IgM diagnostic design utilized a system comprising a container (e.g., a test tube) containing dried nanoprobe conjugates and a paper strip design containing dried UCON-50-HB-5100 and potassium phosphate (UCON/salt) ATPS components. In this Tube and UCON-based Biomarker Extraction setup (designated as the TUBE), the dried components are designed to be resolubilized in a specific order in which the target is first captured by the conjugates and then concentrated within paper.

Note that the execution of both designs is more difficult than merely dehydrating components and subsequently rehydrating them, as the rehydrated components need to yield the appropriate phase separation conditions. Accordingly, this process was optimized so that it properly integrated with an LFA and demonstrated its ability to improve the LFA limit of detection for infectious disease biomarkers by 10-fold without compromising the accuracy of the test results. To our knowledge, this is the first demonstration of dehydrating ATPS components onto paper to provide a sequential solubilization protocol that permits only the sample to be added to achieve phase separation and concentration of the target.

In certain embodiments methods and devices described herein can be provided for analyte collection, extraction, concentration, and detection for clinical applications. In certain embodiments the methods and devices permit the rapid detection and/or quantification of bacteria, fungi, protozoa, viruses, or other analytes, in biological samples (e.g., oral fluid or tissue sample, urine, blood or blood fraction, cerebrospinal fluid, lymph, tissue biopsies, vaginal samples, and the like), food samples, environmental samples, and the like.

In certain embodiments the assays and devices provided herein are accurate, sensitive, portable, disposable, and well suited to use at point of care, for in field environmental testing, field food testing, and the like, with minimal training or equipment.

ARROW Format Assays.

One illustrative, but non-limiting embodiments of a dehydrated ATPS diagnostic device (e.g., a dehydrated PEG/salt ATPS diagnostic device) is shown in FIGS. 5A and 5B. As illustrated, in various embodiments, this device is comprised of two major components: the ATPS Rehydration and Resolubilization Optimized Wick (ARROW) and the standard lateral flow immunoassay (LFA). In the illustrated embodiments, the ARROW consisted of several paper sheets (e.g., fiberglass sheets) layered together. However, it will be recognized that in certain embodiments a single sheet can be used, or in certain embodiments, the wick comprises at least 2, or at least 3, or at least 4, or at least 5, or at least 6, or at least 7, or at least 8, or at least 9, or at least 10, or at least 15, or at least 20 layers of the paper.

Considering that the function of the ATPS is to concentrate the target pathogen, it was desirable that the ARROW was able to wick up a large volume of sample solution. In the illustrated embodiment 15% (w/w) of salt (e.g., potassium phosphate) was dehydrated in the upstream portion of each paper (e.g., fiberglass) sheet, while 10% (w/w) polymer (e.g., PEG 8000) was dehydrated in the downstream portion of each paper sheet. However, it will be recognized that these quantities can be varied as described below.

In certain embodiments a blank space is left between the dehydrated polymer (e.g., PEG) and the tip of the sheet to allow for collection of the polymer-poor phase that contains the concentrated analyte (e.g., pathogen). In certain embodiments the downstream tip of each sheet can tapered (e.g., to form a point), which facilitates proper transition of the liquid into the LFA (e.g., into a conjugate pad of an LFA).

In the illustrated embodiment the LFA portion of the diagnostic consisted of a conjugate pad, containing the colorimetric indicator, connected to a nitrocellulose membrane with printed primary and secondary antibodies (e.g., to provide an indicator line and a control line), and followed by an absorbent pad. It will be recognized, however, that the colorimetric indicator need not be provided in the LFA. Thus, in certain embodiments the colorimetric indicator can be provided in a region of the wick (ARROW). It will also be recognized that the indicator need not be a colorimetric indicator and in various embodiments the indicator can simply comprise, inter alia, a nanoconjugate comprising an indicator moiety attached to an analyte binding moiety that binds to the analyte to be detected, e.g., as described below.

In certain embodiments the ARROW is configured to provide fluid communication to an LFA. Thus, for example, the LFA portion interfaced with the ARROW by fitting a small upstream portion of the conjugate pad perpendicularly into a slit that had been cut in the ARROW.

In the illustrated embodiment, the ARROW was designed to concentrate a biomarker capable of partitioning to a single phase on its own. Since the C. trachomatis whole bacteria is relatively large (0.8 to 1 μm), it can partition extremely to the PEG-poor phase without intervention.

It will be noted that while FIGS. 5A and 5B illustrates the wick integrated with an LFA, it will be recognized that, in certain embodiments, the wick can be utilized separately from the LFA in combination with a separate LFA or with other assay systems, or simply as an analyte reagent concentrator alone.

While the ARROW system described above provides for ATPS components in separate regions to permit sequential rehydration, in certain embodiments it is desirable for the first component and the second component to be rehydrated substantially simultaneously as in various embodiments of the TUBE format assays described below. Accordingly, in certain embodiments, the wick comprises the first component and the second component of the ATPS provided in dried form in substantially the same region so that when contacted with a fluid sample, both components are rehydrated at substantially the same time.

In view of the forgoing, numerous variations of the ARROW comprising different papers, different ATPS components, different nanoconjugates, configured to detect different analytes, and the like will be available to one of skill in the art.

TUBE Format Assays.

Many infectious disease biomarker targets, such as the HIV antibodies typically detected in HIV rapid tests, are smaller in scale and do not partition extremely to a single phase. Therefore, another strategy can be utilized to concentrate these biomarkers. Previously, our group demonstrated that the gold nanoparticle conjugates typically used in LFA can be added directly into an ATPS, where they partition extremely to the polymer-poor phase in a polymer/salt ATPS. This partitioning can be exploited for performing an ATPS where the analyte does not partition extremely to a single phase.

In this format, a nanoconjugate comprising a binding moiety that binds to the analyte attached to an indicator, e.g., a gold nanoparticle, is added to the sample solution and allowed to bind the target analyte present in solution before phase separation occurs. After the onset of phase separation, the large nanoconjugate/target complexes partition to a single phase, e.g., a UCON-poor phase in a UCON/salt ATPS, thus concentrating the target into the single phase.

Extraction of the partitioned complexes and application to the LFA yielded improvements in the detection limit of the bound targets. In this study, we focused on incorporating this mechanism into the dehydrated format to concentrate smaller targets, using a human IgM antibody (970 kDa, or approximately 37 nm in diameter) as a model biomarker target.

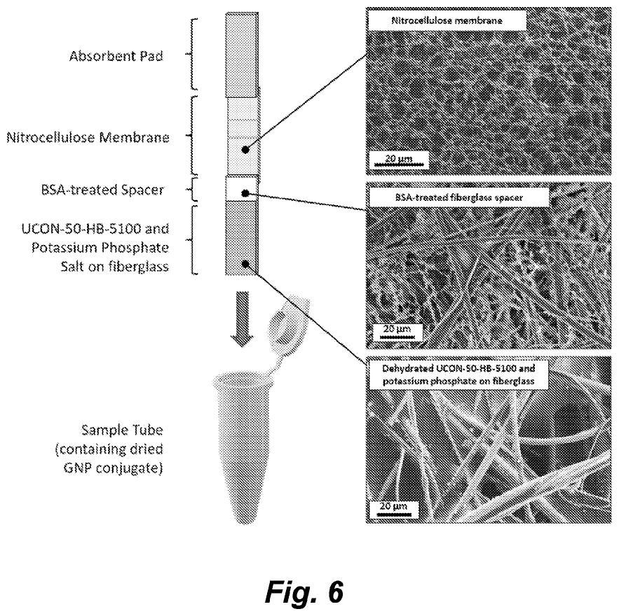

One embodiment of this approach is shown in the “TUBE” design illustrated in FIG. 6. As illustrated, the “TUBE” system is comprised of two main components: 1) a sample tube; and 2) a test strip that comprises ATPS (e.g., UCON/salt) pads connected to the standard LFA. In this design, it is desirable that the nanoconjugates access the entire sample solution and bind to the target prior to the ATPS concentration step. It is also important that after binding the target, the nanoconjugates access the dehydrated ATPS region at the same time in order to maximize the nanoconjugates (e.g., gold nanoparticle(s) (GNP(s)) that become concentrated into the resulting polymer-poor (e.g., UCON-poor) leading front. One approach to achieve these design criteria was to dry the nanoconjugates and store them in powder form housed in a sample tube (e.g., a microcentrifuge tube). In this case, the liquid sample is first added into the tube, which results in the nanoconjugates resolubilizing and bind any analyte (e.g., human IgM) present in the sample. Next, the test strip is added into the sample tube, and the nanoconjugates (e.g., GNPs) collectively wick up the test strip, first making contact with the ATPS region (e.g., UCON/salt pad). When this occurs, the dehydrated ATPS components (e.g., UCON/salt mixture) are rehydrated by the wicking solution, inducing the formation and separation of the of the ATPS (e.g., into UCON-rich and the UCON-poor phases). The analyte-bound nanoconjugates (e.g., GNPs) are concentrated in the newly-formed polymer-poor (e.g., UCON-poor) fluid front, while the newly-formed and more viscous polymer-rich (e.g., UCON-rich) region lags behind. A spacer pad that optionally contains one or more reagents (e.g., BSA) to reduce or prevent non-specific binding can ensure even transition of the polymer-poor (e.g., UCON-poor) phase into the LFA detection region and prevent or reduce nonspecific binding of the nanoconjugates.

The particular TUBE format shown in FIG. 6 is illustrative and non-limiting. In view of the forgoing, numerous variations of the TUBE format comprising different papers, different ATPS components, different nanoconjugates, configured to detect different analytes, and the like will be available to one of skill in the art.

ATPS and ATPS Components.

In various embodiments the devices described herein are configured to incorporate components of aqueous two-phase systems (ATPS), where the components of the ATPS (a first component and a second component) are provided in a dry form in a wick or as a component of an LFA device. In certain embodiments ATPS components are disposed so that they rehydrate sequentially upon contact with a sample. The ATPS components are provided in sufficient quantity that when rehydrated by a fluid sample (e.g., an aqueous sample) containing sample material to be assay for a target analyte, the components form a mixed phase solution that partitions and concentrates the target analyte(s) and/or analyte/nanoconjugate complexes.

In some embodiments, the ATPS components, when rehydrated, comprise two aqueous solutions, a first phase solution and a second phase solution that effectively mix to form a mixed phase solution and then partition as the solution moves through the paper. In some embodiments, the mixed phase solution is a homogeneous solution, while in certain other embodiments the hydrated first phase solution and the second phase solution are immiscible. In some embodiments, the first phase solution and the second phase solution are immiscible, but domains of the hydrated first phase solution mix with domains of the hydrated second phase solution. In some embodiments, the degree of miscibility is driven by changes in temperature, and/or changes in the concentrations of the different components, such as salt. In some embodiments, the first/second phase can comprise components, such as, micelles, salts, and/or polymers. In some embodiments, the target analyte (e.g., biomolecule, bacterium (or fragment thereof), fungus (or fragment thereof), or virus, and the like) in contact with the ATPS, distributes, partitions, and/or concentrates preferentially into the resolubilized first phase over the second phase, or vice versa, based on its physical and chemical properties, such as size, shape, hydrophobicity, and charge. In some embodiments, the target analyte (e.g. a bacterium, fungus, virus, etc.) partitions predominantly (or extremely) into the rehydrated first or second phase solution of the ATPS, and therefore concentrates in the ATPS. In some embodiments, the target analyte is concentrated by adjusting the ratio of volumes between the rehydrated first phase solution and the rehydrated second phase solution. In some embodiments, the target analyte is concentrated by reducing the volume of the phase in which the analyte partitions. By way of illustration, in some embodiments, the target analyte is concentrated by 10-fold in the rehydrated first phase solution, e.g., by using a 1:9 volume ratio of rehydrated first phase solution to rehydrated second phase solution, since the volume of the phase into which the analyte extremely partitions into is 1/10 the total volume.

In some embodiments, other concentrations are obtained by using other ratios. Thus, in some embodiments the ratio of the rehydrated first phase solution to the rehydrated second phase solution comprises a ratio of about 1:1, about 1:2, about 1:3, about 1:4, about 1:5, about 1:6, about 1:7, about 1:8, about 1:9, or about 1:10. In some embodiments the ratio of the rehydrated first phase solution to the rehydrated second phase solution comprises a ratio of about 1:20, about 1:30, about 1:40, about 1:50, about 1:60, about 1:70, about 1:80, about 1:90, or about 1:100. In some embodiments the ratio of the rehydrated first phase solution to the rehydrated second phase solution comprises a ratio of about 1:200, about 1:300, about 1:400, about 1:500, about 1:600, about 1:700, about 1:800, about 1:900, or about 1:1000.

In some embodiments the ratio of the rehydrated second phase solution to the rehydrated first phase solution comprises a ratio of about 1:1, about 1:2, about 1:3, about 1:4, about 1:5, about 1:6, about 1:7, about 1:8, about 1:9, or about 1:10. In some embodiments the ratio of the rehydrated second phase solution to the rehydrated first phase solution comprises a ratio of about 1:20, about 1:30, about 1:40, about 1:50, about 1:60, about 1:70, about 1:80, about 1:90, or about 1:100. In some embodiments the ratio of the rehydrated second phase solution to the rehydrated first phase solution comprises a ratio of about 1:200, about 1:300, about 1:400, about 1:500, about 1:600, about 1:700, about 1:800, about 1:900, or about 1:1000.

In some embodiments, the analyte partitions substantially evenly between the rehydrated first phase solution and rehydrated second phase solution, preventing concentration of the analyte. In such systems, concentration of the target analyte can be achieved by introducing an additional component, such as a probe (e.g., an indicator moiety attached to a binding moiety that binds to the analyte to be detected) that captures the target analyte, where the probe partitions predominantly into one phase, thereby enhancing the partitioning behavior of the target analyte to enable concentration.

In some embodiments, the rehydrated first/second phase solution comprises a micellar solution. In some embodiments, the micellar solution comprises a nonionic surfactant. In some embodiments, the micellar solution comprises a detergent. In some embodiments, the micellar solution comprises Triton-X. In some embodiments, the micellar solution comprises a polymer similar to Triton-X, such as Igepal CA-630 and Nonidet P-40, and the like, by way of non-limiting example. In some embodiments, the micellar solution consists essentially of Triton-X.

In some embodiments, the rehydrated micellar solution has a viscosity (at room temperature (˜25° C.) of about 0.01 centipoise to about 5000 centipoise, about 0.01 centipoise to about 4500 centipoise, about 0.01 centipoise to about 4000 centipoise, about 0.01 centipoise to about 3500 centipoise, about 0.01 centipoise to about 3000 centipoise, about 0.01 centipoise to about 2500 centipoise, about 0.01 centipoise to about 2000 centipoise, about 0.01 centipoise to about 1500 centipoise, about 0.01 centipoise to about 1000 centipoise, or about 0.01 centipoise to about 500 centipoise. In some embodiments, the micellar solution has a viscosity at room temperature of about 0.01 centipoise to about 450 centipoise, about 0.01 centipoise to about 400 centipoise, about 0.01 centipoise to about 350 centipoise, about 0.01 centipoise to about 300 centipoise, about 0.01 centipoise to about 250 centipoise, about 0.01 centipoise to about 200 centipoise, about 0.01 centipoise to about 150 centipoise, or about 0.01 centipoise to about 100 centipoise.

In some embodiments, the rehydrated first/second phase solution comprises a polymer (e.g., polymer solution). In certain embodiments the polymer comprises one or more polymers selected from the group consisting of polyethylene glycol (PEG), ethylene/propylene copolymer (e.g., a UCON™ polymer), propylene glycol (PPG), methoxypolyethylene glycol, polyvinyl pyrrolidone, and the like. In certain embodiments, the polymer is a polyethylene glycol (PEG). In various embodiments, the PEG may have a molecular weight between 1000 and 100,000. In certain embodiments, the PEG comprises PEG-4600, PEG-8000, or PEG-20,000. In certain embodiments, the polymer is polypropylene glycol (PPG). In various embodiments, the PPG may have a molecular weight between 100 and 10,000. In certain embodiments, the PPG comprises PPG 425. In certain embodiments, the polymer is dextran. In various embodiments, the dextran may have a molecular weight between 1000 and 1,000,000. In certain embodiments, the dextran comprises dextran 6000, dextran 9000, dextran-35,000, or dextran-200,000. In certain embodiments the polymer comprises an ethylene/propylene copolymer (e.g., a UCON™ polymer). Illustrative, but non-limiting ethylene/propylene copolymers include, but are not limited to UCON™ 50-HB-5100, UCON™ 50-HB-3520, UCON™ 50-HB-2000, UCON™ 50-HB-660, UCON™ 50-HB-400, UCON™ 50-HB-260, UCON™ 50-HB-170, UCON™ 50-HB-100, UCON™ 60-H-5300, UCON™ 60-H2300, UCON™ 60-H-1600, UCON™ 60-H-1100, UCON™ 60-H-760, UCON™ 60-H-340, UCON™ 75-H-9500, UCON™ 75-H-1400, UCON™ 75-H-450, and the like.

In some embodiments, the rehydrated polymer solution comprises a polymer solution that is about 0.01% w/w polymer, or about 0.05% w/w polymer, or about 0.1% w/w polymer, or about 0.15% w/w polymer, or about 0.2% w/w polymer, or about 0.25% w/w polymer, or about 0.3% w/w polymer, or about 0.35% w/w polymer, or about 0.4% w/w polymer, or about 0.45% w/w polymer, or about 0.5% w/w polymer, or about 0.55% w/w polymer, or about 0.6% w/w polymer, or about 0.65% w/w polymer, or about 0.7% w/w polymer, or about 0.75% w/w polymer, or about 0.8% w/w polymer, or about 0.85% w/w polymer, or about 0.9% w/w polymer, or about 0.95% w/w polymer, or about 1% w/w polymer. In some embodiments, the polymer solution comprises a polymer solution that is about 1% w/w polymer, or about 2% w/w polymer, or about 3% w/w polymer, or about 4% w/w polymer, or about 5% w/w polymer, or about 6% w/w polymer, or about 7% w/w polymer, or about 8% w/w polymer, or about 9% w/w polymer, or about 10% w/w polymer, or about 11% w/w polymer, or about 12% w/w polymer, or about 13% w/w polymer, or about 14% w/w polymer, or about 15% w/w polymer, or about 16% w/w polymer, or about 17% w/w polymer, or about 18% w/w polymer, or about 19% w/w polymer, or about 20% w/w polymer, or about 21% w/w polymer, or about 22% w/w polymer, or about 23% w/w polymer, or about 24% w/w polymer, or about 25% w/w polymer, or about 26% w/w polymer, or about 27% w/w polymer, or about 28% w/w polymer, or about 29% w/w polymer, or about 30% w/w polymer, or about 31% w/w polymer, or about 32% w/w polymer, or about 33% w/w polymer, or about 34% w/w polymer, or about 35% w/w polymer, or about 36% w/w polymer, or about 37% w/w polymer, or about 38% w/w polymer, or about 39% w/w polymer, or about 40% w/w polymer, or about 41% w/w polymer, or about 42% w/w polymer, or about 43% w/w polymer, or about 44% w/w polymer, or about 45% w/w polymer, or about 46% w/w polymer, or about 47% w/w polymer, or about 48% w/w polymer, or about 49% w/w polymer, or and about 50% w/w polymer. In some embodiments, the polymer solution comprises a polymer solution that is about 10% w/w polymer, or about 20% w/w polymer, or about 30% w/w polymer, or about 40% w/w polymer, or about 50% w/w polymer, or about 60% w/w polymer, or about 70% w/w polymer, or about 80% w/w polymer, or about 90% w/w polymer. In some embodiments, the polymer solution comprises a polymer solution that is about 10% w/w polymer to about 80% w/w polymer. In some embodiments, the rehydrated polymer solution comprises a polymer solution that is about 1% w/w to about 30% w/w, or from about 5% w/w up to about 25% w/w, or from about 10% w/w up to about 25% w/w, or from about 10% w/w up to about 20% w/w polymer.

In some embodiments, the rehydrated first and/or second phase solution comprises a salt and thereby forms a salt solution. In some embodiments, the target analyte (e.g., bacterium, fungus, virus, etc.) and/or a probe-analyte complex partitions into the salt solution. In certain embodiments the salt solution comprises a kosmotropic salt. In some embodiments the salt solution comprises a chaotropic salt. In some embodiments, the salt comprises one or more of a magnesium salt, a lithium salt, a sodium salt, a potassium salt, a cesium salt, a zinc salt, and an aluminum salt. In some embodiments, the salt comprises a bromide salt, an iodide salt, a fluoride salt, a carbonate salt, a sulfate salt, a citrate salt, a carboxylate salt, a borate salt, or a phosphate salt. In some embodiments, the salt is potassium phosphate. In some embodiments, the salt is ammonium sulfate.

In some embodiments, the rehydrated salt solution comprises a salt solution comprising about 0.01% w/w salt, or about 0.05% w/w salt, about 0.1% w/w salt, or about 0.15% w/w salt, or about 0.2% w/w salt, or about 0.25% w/w salt, or about 0.3% w/w salt, or about 0.35% w/w salt, or about 0.4% w/w salt, or about 0.45% w/w salt, or about 0.5% w/w salt, or about 0.55% w/w salt, or about 0.6% w/w salt, or about 0.65% w/w salt, or about 0.7% w/w salt, or about 0.75% w/w salt, or about 0.8% w/w salt, or about 0.85% w/w salt, or about 0.9% w/w salt, or about 0.95% w/w salt, or about or about 1% w/w salt. In some embodiments, the rehydrated salt solution comprises a salt solution that is about 1% w/w salt, or about 2% w/w salt, or about 3% w/w salt, or about 4% w/w salt, or about 5% w/w salt, or about 6% w/w salt, or about 7% w/w salt, or about 8% w/w salt, or about 9% w/w salt, or about 10% w/w salt, or about 11% w/w salt, or about 12% w/w salt, or about 13% w/w salt, or about 14% w/w salt, or about 15% w/w salt, or about 16% w/w salt, or about 17% w/w salt, or about 18% w/w salt, or about 19% w/w salt, or about 20% w/w salt, or about 21% w/w salt, or about 22% w/w salt, or about 23% w/w salt, or about 24% w/w salt, or about 25% w/w salt, or about 26% w/w salt, or about 27% w/w salt, or about 28% w/w salt, or about 29% w/w salt, or about 30% w/w salt, or about 31% w/w salt, or about 32% w/w salt, or about 33% w/w salt, or about 34% w/w salt, or about 35% w/w salt, or about 36% w/w salt, or about 37% w/w salt, or about 38% w/w salt, or about 39% w/w salt, or about 40% w/w salt, or about 41% w/w salt, or about 42% w/w salt, or about 43% w/w salt, or about 44% w/w salt, or about 45% w/w salt, or about 46% w/w salt, or about 47% w/w salt, or about 48% w/w salt, or about 49% w/w salt, or and about 50% w/w. In some embodiments, the rehydrated salt solution comprises a salt solution that ranges from about 0.1% w/w to about 40% w/w, or from about 1% w/w up to about 30% w/w, or from about 5% w/w up to about 25% w/w, or from about 10% w/w up to about 20% w/w. In some embodiments, the rehydrated salt solution comprises a salt solution that is about 0.1% w/w to about 10%. In some embodiments, the salt solution is about 1% w/w to about 10%.

In some embodiments, the rehydrated first/second phase solution comprises a solvent that is immiscible with water. In some embodiments, the solvent comprises a non-polar organic solvent. In some embodiments, the solvent comprises an oil. In some embodiments, the solvent comprises pentane, cyclopentane, benzene, 1,4-dioxane, diethyl ether, dichloromethane, chloroform, toluene, or hexane.

In some embodiments, the rehydrated first phase solution comprises a micellar solution and the rehydrated second phase solution comprises a polymer. In some embodiments, the rehydrated second phase solution comprises a micellar solution and the rehydrated first phase solution comprises a polymer. In some embodiments, the rehydrated first phase solution comprises a micellar solution and the rehydrated second phase solution comprises a salt. In some embodiments, the rehydrated second phase solution comprises a micellar solution and the rehydrated first phase solution comprises a salt. In some embodiments, the micellar solution is a Triton-X solution. In some embodiments, the rehydrated first phase solution comprises a first polymer and the rehydrated second phase solution comprises a second polymer. In some embodiments, the rehydrated first/second polymer comprises polyethylene glycol and/or dextran. In some embodiments, the rehydrated first phase solution comprises a salt and the rehydrated second phase solution comprises a salt. In some embodiments, the rehydrated second phase solution comprises a polymer and the rehydrated first phase solution comprises a salt. In some embodiments, the first phase solution comprises polyethylene glycol and the second phase solution comprises potassium phosphate. In some embodiments, the second phase solution comprises polyethylene glycol and the first phase solution comprises potassium phosphate. In some embodiments, the first phase solution comprises a salt and the second phase solution comprises a salt. In some embodiments, the first phase solution comprises a kosmotropic salt and the second phase solution comprises a chaotropic salt. In some embodiments, the second phase solution comprises a kosmotropic salt and the first phase solution comprises a chaotropic salt.

In some embodiments, the rehydrated first phase solution comprises a Component 1 of Table 1 and the rehydrated second phase solution comprises a Component 2 of Table 1. In some embodiments, the rehydrated second phase solution comprises a Component 1 of Table 1 and rehydrated the second phase solution comprises a Component 2 of Table 1.

In some embodiments, before drying, the components of Table 1 are suspended or dissolved in a buffer. In some embodiments, before drying the components of Table 1 are suspended/dissolved in a buffer compatible with a biological system from which the sample was derived. In some embodiments, before drying the components of Table 1 are suspended/dissolved in a saline solution. In some embodiments, before drying the components of Table 1 are suspended/dissolved in PBS. In some embodiments, the components of Table 1 before drying are suspended/dissolved in water. In some embodiments, the components of Table 1 before drying are suspended/dissolved in a biological fluid.

TABLE 1
Illustrative aqueous two-phase polymer/salt
extraction/concentration systems.

| Component 1 | Component 2 |
|---|---|
| Potassium phosphate Sodium sulfate Magnesium sulfate Ammonium sulfate Sodium citrate Magnesium chloride Magnesium citrate Magnesium phosphate Sodium chloride Potassium citrate Potassium carbonate | Polyethylene glycol (PEG) Ethylene/propylene copolymer Propylene glycol (PPG) Methoxypolyethylene glycol Polyvinyl pyrrolidone |
| Potassium phosphate Sodium sulfate Magnesium sulfate Ammonium sulfate Sodium citrate Magnesium chloride Magnesium citrate Magnesium phosphate Sodium chloride Potassium citrate Potassium carbonate | Ethylene/propylene copolymer (e.g, UCON ™ 50-HB-5100, UCON ™ 50-HB-3520, UCON ™ 50-HB-2000, UCON ™ 50-HB-660, UCON ™ 50-HB-400, UCON ™ 50-HB-260, UCON ™ 50-HB-170, UCON ™ 50-HB-100, UCON ™ 60-H-5300, UCON ™ 60-H2300, UCON ™ 60-H-1600, UCON ™ 60-H-1100, UCON ™ 60-H-760, UCON ™ 60-H-340, UCON ™ 75-H-9500, UCON ™ 75-H-1400, UCON ™ 75-H-450, etc.) |
| Potassium phosphate Sodium sulfate Magnesium sulfate Ammonium sulfate Sodium citrate Magnesium chloride Magnesium citrate Magnesium phosphate Sodium chloride Potassium citrate Potassium carbonate | Polyethylene glycol (PEG) |
| Potassium phosphate | Ethylene/propylene copolymer |
| Potassium phosphate | Polyethylene glycol (PEG) |
| Potassium phosphate | Propylene glycol (PPG) |
| Potassium phosphate | Methoxypolyethylene glycol |
| Potassium phosphate | Polyvinyl pyrrolidone |

It will be noted that UCON™ 50-HB polymers comprise ethylene/propylene copolymers produced by reacting an equal amount by weight of ethylene oxide and propylene oxide with butyl alcohol using an alkali catalyst at temperatures from about 100° C. to about 150° C. The resulting UCON™ 50-HB is a random copolymer with the general structure:

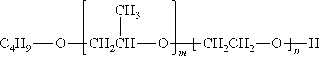

It will be recognized that the above-described ATPS systems and components are illustrative and non-limiting. Using the teachings provided herein, numerous other ATPS systems and components will be available to one of skill in the art.

Lateral Flow Assay.

In certain embodiments, the wick described herein is configured to work in conjunction with a lateral-flow assay (LFA) and the systems described herein are configured to provide a lateral flow assay for the detection of one or more target analyte(s). The LFA typically comprises a porous matrix (e.g., a paper) into which are disposed sample and assay components, e.g., as described above. The porous matrix is configured to and has porosity sufficient to allow the assay reagents to flow through the porous matrix when the components are in a fluid phase. Such porous LFA devices are be referred to as paper or paper fluidic devices and these terms are used interchangeably.

Lateral flow assays (LFAs) are based on the use of a porous matrix (e.g., a paper), such as pieces of porous paper, microstructured polymer, sintered polymer, and the like. The porous matrix is selected for, inter alia, its capacity to transport fluid through the matrix, e.g., via capillary action. A typical LFA comprises a sample receiving zone (e.g., a sample pad) that can act as a sponge and hold the applied sample fluid. The applied/received fluid migrates through the LFA to a conjugate zone (e.g., a conjugate pad) that, in certain embodiments contains a nanoconjugate (e.g., an indicator attached to a moiety (e.g., an antibody) that binds the target analyte that is to be detected. When the fluid migrates to the conjugate zone, the nanoconjugate binds to the analyte in the sample if present forming a nanoconjugate/analyte complex. It will be noted that in certain embodiments described herein the sample can be contacted to nanoconjugates outside a test strip (see, e.g., the TUBE format described herein) in which case the LFA need not incorporate a conjugate zone.

The nanoconjugate binds to the analyte while flowing through the porous matrix comprising the LFA. The LFA typically comprises a detection zone comprising immobilized moieties (capture moieties) that bind to the analyte/nanoconjugate complex and thereby immobilize the analyte/nanoconjugate complex. Often the immobilized moieties are arranged to form a line or strip. As the analyte/nanoconjugate complex accumulates at a line in a detection zone a detectable signal (e.g., a visual chromogenic signal) is produced indicating the presence of the analyte. In certain embodiments the LFA additionally comprises a control zone containing capture moieties that bind the nanoconjugate and the nanoconjugate analyte complex to provide a positive signal indicating that reagents have passed through the detection zone.

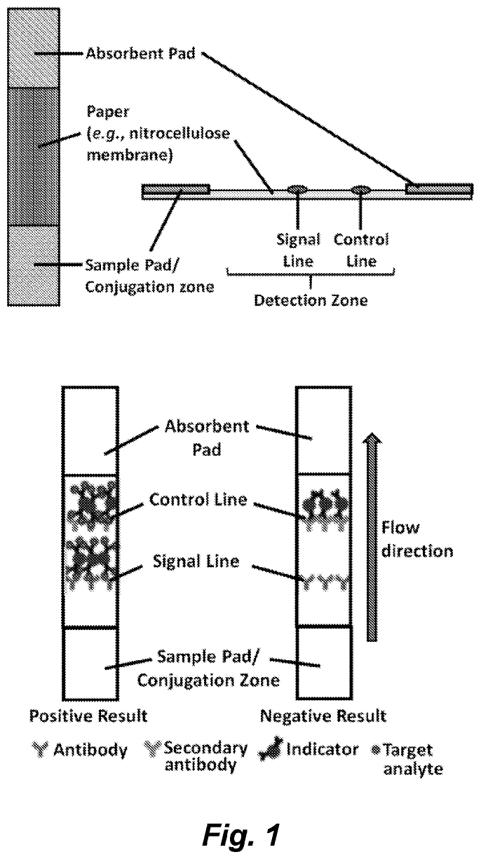

In certain embodiments after passing these reaction zones the fluid enters the final porous material, e.g., an absorbent zone, that simply acts as a waste container. In various embodiments the LFAs can be configured to operate as either competitive or sandwich assays. An LFA is schematically illustrated in Figure. 1.

Accordingly, in various embodiments the lateral flow assay comprises a porous substrate (e.g., a paper), a sample receiving zone disposed on or in the paper, and a detection zone disposed on or in the paper where the detection zone comprises at least a first test line, and, optionally a second test line, and in certain embodiments optionally a third test line. In illustrative, but non-limiting embodiments, the test line(s) can be defined by row(s) of immobilized binding moieties (e.g., antibodies) that capture the analyte (e.g., an analyte/indicator complex) when such analyte is present. In certain embodiments the lateral flow assay additionally comprises a conjugation zone containing the indicator attached to a moiety that binds the target analyte. The lateral flow device can additionally comprise a control line and/or an absorbent pad (e.g., sink).

Sample Receiving Zone

In certain embodiments the LFA devices described herein comprise a sample receiving zone for application/receiving of the biological sample. In certain embodiments the sample receiving zone comprises a sample pad disposed on or in the paper substrate. In certain embodiments the sample pad can act as a filter that can remove debris, contaminants, and mucus from the collected fluid. It can also store dried reagents, and when rehydrated, these reagents can (i) adjust the solution for optimal detection conditions (pH, ionic strength, etc.); and (ii) break down mucus, glycoproteins, and other viscous materials in the collected specimen that may affect detection.

Illustrative materials for the sample pad include, but are not limited to, cellulose, nitrocellulose, fiberglass, cotton, woven or nonwoven paper, etc. Reagents on the pad may include, but are not limited to, surfactants such as Triton X-100, Tween 20, or sodium dodecyl sulfate, etc.; polymers such as polyethylene glycol, poloxamer, polyvinylpyrrolidone (PVP), etc.; buffers such as phosphate-buffered saline, 4-(2-hydroxyethyl)-1-piperazineethanesulfonic acid (HEPES), Tris(hydroxymethyl)aminomethane (Tris), sodium borate, TRICINE, etc.; proteins such as albumin, etc.; enzymes such as protease, etc.; salts such as sodium chloride, sodium phosphate, sodium cholate, potassium phosphate, etc. In various embodiments these reagents can be applied to the sample pad by (i) soaking the paper material in the reagent solution, or (ii) through wicking the membrane via capillary flow. The treated sample pad can be dried by (i) air drying (let sit in room temperature); (ii) baking (place in high temperature using an oven or heating device); (iii) vacuum; or (iv) lyophilization.

Conjugation Zone

In certain embodiments the LFA devices described herein can comprise a conjugation zone for mixing the sample with a nanoconjugate (e.g., an indicator attached to a moiety that binds to the target analyte). In certain embodiments the conjugation zone comprises a conjugate pad. In certain embodiments the conjugation zone, when present can contain dehydrated indicators (e.g., colorimetric indicators, fluorescent indicators, radioactive indicators, magnetic indicators, etc.) decorated with binding moieties that bind the target analyte(s). In certain embodiments the binding moieties are specific binding moieties that have high affinity towards the target analyte(s). When the sample solution reaches the conjugate pad, the indicators (e.g., colorimetric indicators) are rehydrated. The binding moieties on the indicators can then bind to the analyte and the resulting complexes can flow to the detection zone. In certain embodiments the indicators can comprise colorimetric indicators that can comprise metallic particles such as gold, silver particles, polymeric particles such as latex beads, and polystyrene particles encapsulating visible or fluorescent dyes. Illustrative materials material for the conjugation zone (e.g., conjugate pad) include, but are not limited to, cellulose, nitrocellulose, fiberglass, cotton, woven or nonwoven paper, etc. In certain embodiments the colorimetric indicators can be applied and dehydrated onto the pad as described above.

Detection Zone

In certain embodiments the LFA comprises a detection zone (e.g., a reaction pad), that can comprise immobilized reagents that capture an analyte/nanoconjugate complex (e.g. for test signal) or that capture an a nanoconjugate without analyte and an analyte nanoconjugate complex. Capture of the analyte/nanoconjugate complex and/or the nanoconjugate without analyte can produce a detectable signal (e.g., a visual signal) to indicate the presence or absence or quantity of the target analyte(s) at particular test lines and/or to provide a control signal at a control line. Illustrative materials for the detection zone include, but are not limited to cellulose, nitrocellulose, fiberglass, cotton, woven or nonwoven paper etc.

In certain embodiments for a lateral-flow test strip, the reagents in the detection zone are immobilized in the form of lines perpendicular to the direction of flow to ensure all samples can interact with the immobilized reagents. The concentrations of the reagents can be optimized to control the signal intensities, and thus, control the sensitivity of the assay. For example, a semi-quantitative assay can be designed by immobilizing multiple lines of the same reagent with various concentrations. Each line therefore will yield signals only when a specific concentration of target biomolecules is reached. The concentration of the target biomolecules can then be interpreted by counting the number of lines that are visible, e.g., as described above.

Absorbent Pad/Sink

In certain embodiments the lateral flow device comprises an absorbent pad disposed downstream from the detection zone and when said control line is present the absorbent pad is disposed downstream from the control line. In certain embodiments the sink, when present, can comprise an absorbent pad that collect excess fluid and prevents back-flow which can affect the test performance. Illustrative materials for the sink include, but are not limited to cellulose, nitrocellulose, fiberglass, cotton, woven and nonwoven paper etc.

Papers Comprising the LFA and/or Wick.

In various embodiments the LFA and/or wick (ARROW) described herein comprise one or more papers that provide a porous matrix through which the ATPS and/or sample solution can flow. The porous matrix is configured to and has porosity sufficient to allow the ATPS or components thereof to flow through the porous matrix when the ATPS or components thereof are in a fluid phase. Such porous LFA are referred to herein as paper or paper fluidic devices and these terms are used interchangeably.

The term “paper”, as used herein, is not limited to thin sheets from the pulp of wood or other fibrous plant substances although, in certain embodiments the use of such papers in the devices described herein is contemplated. Papers more generally refer to porous materials often in sheet form, but not limited thereto that allow a fluid to flow through.

In some embodiments, the porous matrix is sufficiently porous to allow the mixed phase solution, first phase solution and/or second phase solution of the ATPS, and/or target analyte, to flow through the LFA. In some embodiments, the porous matrix is sufficiently long and/or deep enough for the mixed phase solution, first phase solution and/or second phase solution, and/or target analyte, to flow vertically and/or horizontally through the LFA or spot assay device. In some embodiments, the first phase solution flows through the porous matrix at a first rate and the second phase solution flows through the porous matrix at a second rate, where the first rate and the second rate are different. In some embodiments of the LFA or spot assay the porous matrix comprises inter alia a material such as a sintered glass ceramic, a mineral, cellulose, a fiberglass, a nitrocellulose, polyvinylidene fluoride, a nylon, a charge modified nylon, a polyethersulfone, combinations thereof, and the like.

Sandwich Assay

In some embodiments, the LFA is configured to provide or run a sandwich assay (see e.g., FIG. 1). In some embodiments, the sandwich assay comprises a capture moiety (e.g., an antibody) that binds the target analyte, e.g., when the analyte is a component of a nanoconjugate/analyte complex. In some embodiments, the device comprises a nanoconjugate (e.g., an indicator attached to a binding moiety (e.g., an antibody) that binds to the analyte of interest). In some embodiments, the indicator provides a detectable property (colorimetric, fluorescent, radioactive, etc.). In some embodiments, the indicator is added to the sample before application to the device and binds the target analyte to form a probe-analyte complex, e.g., in the TUBE systems described herein. In some embodiments, the indicator can be combined with the sample in a conjugation zone in the LFA device after the sample is added to the device and binds the target analyte to form a probe-analyte complex (e.g., in certain embodiments of the ARROW methods described herein).

The nanoconjugate/analyte complex flows through the LFA or through the flow-through device towards the absorbent pad. In some embodiments, the target analyte of the indicator-analyte complex binds to the capture moiety. In some embodiments, the capture moiety is immobilized on a test line or a test region and the nanoconjugate/analyte complex becomes immobilized on the test line or in the test region. In some embodiments, the nanoconjugate comprises a colorimetric moiety (e.g., a gold nanoparticle), and the test line or test region will exhibit a strong color (e.g. detectable signal) as the nanoconjugate/analyte complex accumulates at the test line or in the test region, indicating a positive result. In some embodiments, there is no target analyte present in the sample, and the nanoconjugate of the nanoconjugate/analyte complex does not interact with the capture moiety, and the absence of the test line or signal in the test region indicates a negative result at that test line. In some embodiments, the LFA comprises a nanoconjugate capture moiety on a control line (or in a control region) that interacts directly with the indicator and/or the binding moiety comprising the nanoconjugate, and thus, regardless of the presence of the target analyte in the sample, the nanoconjugate binds to the nanoconjugate capture moiety and accumulates on the control line or in the control region. In some embodiments, the nanoconjugate capture moiety is a secondary antibody that binds the binding moiety, wherein the binding moiety is a primary antibody that binds that target analyte. In some embodiments, the nanoconjugate becomes immobilized and detected on the control line or in the control region, indicating a valid test. In some embodiments, a positive result (e.g. target analyte is present in sample) is indicated by a detectable signal at the test line(s) as described above. In some embodiments, a negative result is indicated by a detectable signal at the control line or in the control region in the absence of test line signal(s) as described above.

Nanoconjugates (Probes).

In certain embodiments the systems and/or devices described herein and/or the methods described herein utilize a nanoconjugate (probe), where the nanoconjugate comprises an indicator moiety attached to an analyte binding moiety that binds the target analyte to form a nanoconjugate/analyte complex.

Indicator Moiety Comprising the Nanoconjugate.

In some embodiments, the indicator moiety comprising the nanoconjugate comprises one or more of a synthetic polymer, a metal, a mineral, a glass, a quartz, a ceramic, a biological polymer, a plastic, and/or combinations thereof. In some embodiments, the nanoconjugate comprises a polymer comprises a polyethylene, polypropylene, nylon (DELRIN®), polytetrafluoroethylene (TEFLON®), dextran and polyvinyl chloride. In some embodiments, the polyethylene is polyethylene glycol. In some embodiments, the polypropylene is polypropylene glycol. In some embodiments, the nanoconjugate comprises a biological polymer that comprises one or more of a collagen, cellulose, and/or chitin. In some embodiments, the nanoconjugate comprises a metal (e.g., that comprises one or more of gold, silver, platinum titanium, stainless steel, aluminum, or alloys thereof). In some embodiments, the nanoconjugate comprises a nanoparticle (e.g., a gold nanoparticle, a silver nanoparticle, etc.).

In some embodiments, the indictor moiety comprises a detectable label. Detectable labels include any composition detectable by spectroscopic, photochemical, biochemical, immunochemical, electrical, optical, or chemical means. Illustrative useful detectable labels include, but are not limited to, fluorescent nanoparticles (e.g., quantum dots (Qdots)), metal nanoparticles, including but not limited to gold nanoparticles, silver nanoparticles, platinum nanoparticles, fluorescent dyes (e.g., fluorescein, Texas Red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In ^{113m}In, ⁹⁷Ru, ⁶²Cu, ⁶⁴¹Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like.

Alternatively or additionally, the indicator moiety is one that can bind to another particle that comprises a detectable label. In some embodiments, the probes provide a detectable signal at the detection zone (e.g., test line, control line, test region, control region). In some embodiments, the indicator moiety provides a detectable property that comprises one or more of a colorimetric label/property, a fluorescent label/property, an enzymatic label/property, a colorigenic label/property, and/or a radioactive label/property. In some embodiments, the probe is a gold nanoparticle and the detectable property is a color. In some embodiments, the color is orange, red or purple.

In some embodiments, the nanoconjugate further comprises a coating. In some embodiments, the coating comprises polyethylene glycol or polypropylene glycol. In some embodiments, the coating comprises polypropylene. In some embodiments, the coating comprises polypropylene glycol. In some embodiments, the coating comprises dextran. In some embodiments, the coating comprises a hydrophilic protein. In some embodiments, the coating comprises serum albumin. In some embodiments, the coating has an affinity for the first phase solution or the second phase solution.

Binding Moiety Comprising the Nanoconjugate.

In some embodiments, the binding moiety comprising the nanoconjugate comprises a molecule that binds the target analyte (e.g., bacterium, fungus, virus, lectin, sugar, protein, DNA, etc.). In some embodiments, the binding moiety is a molecule that specifically binds the target analyte. In some embodiments, “specifically binds” indicates that the molecule binds preferentially to the target analyte or binds with greater affinity to the target analyte than to other molecules. By way of non-limiting example, an antibody will selectively or specifically bind to an antigen against which it was raised. Also, by way of non-limiting example, a DNA molecule will bind to a substantially complementary sequence and not to unrelated sequences under stringent conditions. In some embodiments, “specific binding” can refer to a binding reaction that is determinative of the presence of a target analyte in a heterogeneous population of molecules (e.g., proteins and other biologics). In some embodiments, the binding moiety binds to its particular target analyte and does not bind in a significant amount to other molecules present in the sample.

In some embodiments, the binding moiety comprises an antibody, a lectin, a protein, a glycoprotein, a nucleic acid, monomeric nucleic acid, a polymeric nucleic acid, an aptamer, an aptazyme, a small molecule, a polymer, a lectin, a carbohydrate, a polysaccharide, a sugar, a lipid, or any combination thereof. In some embodiments, the binding moiety is a molecule capable of forming a binding pair with the target analyte.

In some embodiments, the binding moiety is an antibody or antibody fragment. Antibody fragments include, but are not limited to, Fab, Fab′, Fab′-SH, F(ab′)₂, Fv, Fv′, Fd, Fd′, scFv, hsFv fragments, cameloid antibodies, diabodies, and other fragments described above.

In certain embodiments, the binding moiety comprises an aptamer. In some embodiments, the aptamer comprises an antibody-analogue formed from nucleic acids. In some embodiments, the aptamer does not require binding of a label to be detected in some assays, such as nano-CHEM-FET, where the reconfiguration would be detected directly. In some embodiments, the binding moiety comprises an aptazyme. In some embodiments, the aptazyme comprises an enzyme analogue, formed from nucleic acids. In some embodiments, the aptazyme functions to change configuration to capture a specific molecule, only in the presence of a second, specific, analyte.

Nanoconjugates to Facilitate Partitioning of Nanoconjugate/Analyte Complex.

In some embodiments, the target analyte alone partitions preferentially into the first phase solution or second phase solution or interface of the first phase solution and second phase solution. In some embodiments, the target analyte alone partitions extremely into the first phase solution or second phase solution or interface of the first phase solution and second phase solution.

However, in some embodiments, the target analyte alone does not partition preferentially into the first phase solution or second phase solution or interface of the first phase solution and second phase solution. Accordingly, in certain embodiments the nanoconjugate is selected so that the nanoconjugate/analyte complex partitions preferentially or extremely into the first phase solution or into the second phase solution or into the interface of the first phase solution and second phase solution, thereby causing the target analyte (of the nanoconjugate/analyte complex) to partition preferentially or extremely into the first phase solution or into the second phase solution or at the interface of the first phase solution and second phase solution.

In some embodiments, the phrase “partitions preferentially,” when used with respect to the partitioning of the target analyte (or nanoconjugate/analyte complex) to a first/second phase solution of the ATPS, indicates that a greater amount of the target analyte becomes disposed in a preferred phase solution than in another phase solution of the ATPS.

In some embodiments, the phrase “partitions extremely,” when used with respect to the partitioning of the target analyte (or nanoconjugate/analyte complex) to a first/second phase solution of the ATPS, indicates that about 90% or more of the target analyte becomes disposed in a preferred phase solution than in another phase solution of the ATPS.

In some embodiments, a greater amount of the target analyte partitions into the first phase solution. In some embodiments, greater than about 50%, or greater than about 55%, or greater than about 60%, or greater than about 65%, or greater than about 70%, or greater than about 75%, or greater than about 80%, or greater than about 85%, or greater than about 90%, or greater than about 95%, or greater than about 98%, or greater than about 99% of the target analyte partitions into the first phase solution. In some embodiments, greater than about 99%, or greater than about 99.1%, or greater than about 99.2%, or greater than about 99.3%, or greater than about 99.4%, or greater than about 99.5%, or greater than about 99.6%, or greater than about 99.7%, or greater than about 99.8%, or greater than about 99.9% of the target analyte partitions into the first phase solution.

In some embodiments, a greater amount of the analyte partitions into the second phase solution. In some embodiments, greater than about 50%, or greater than about 55%, or greater than about 60%, or greater than about 65%, or greater than about 70%, or greater than about 75%, or greater than about 80%, or greater than about 85%, or greater than about 90%, or greater than about 95%, or greater than about 98%, or greater than about 99% of the target analyte partitions into the second phase solution. In some embodiments, greater than about 99%, or greater than about 99.1%, or greater than about 99.2%, or greater than about 99.3%, or greater than about 99.4%, or greater than about 99.5%, or greater than about 99.6%, or greater than about 99.7%, or greater than about 99.8%, or greater than about 99.9% of the target analyte partitions into the second phase solution.

In some embodiments, a greater amount of the analyte partitions into the interface of the first phase solution and the second phase solution. In some embodiments, greater than about 50%, or greater than about 55%, or greater than about 60%, or greater than about 65%, or greater than about 70%, or greater than about 75%, or greater than about 80%, or greater than about 85%, or greater than about 90%, or greater than about 95%, or greater than about 98%, or greater than about 99% of the target analyte partitions into the interface. In some embodiments, greater than about 99%, or greater than about 99.1%, or greater than about 99.2%, or greater than about 99.3%, or greater than about 99.4%, or greater than about 99.5%, or greater than about 99.6%, or greater than about 99.7%, or greater than about 99.8%, or greater than about 99.9% of the target analyte partitions into the interface.

In some embodiments, the device comprises or is configured to utilize and/or the assay run on the device utilizes one nanoconjugate (probe directed to a single analyte). In some embodiments, the device comprises or is configured to utilize and/or the assay run on the device utilizes at least two different nanoconjugates (each directed to a different analyte), or at least 3 different nanoconjugates, or at least 4 different nanoconjugates, or at least 5 different nanoconjugates, or at least 7 different nanoconjugates, or at least 10 different nanoconjugates, or at least 15 different nanoconjugates, or at least 20 different nanoconjugates.

Sample Collection

In various embodiments the sample to be assayed using the devices and methods described herein comprises a biological sample. Illustrative biological samples include, but are not limited, to biofluids such as blood or blood fractions, urine, lymph, nasal or oral fluids, and the like.

Where the biological sample comprises a tissue, in certain embodiments, the tissue may be lysed, homogenized, and/or ground and, optionally suspended in a sample solution. Where the biological sample comprises a biological fluid, the fluid may be assayed directly or suspended in a sample solution prior to assay. In certain embodiments the sample solution may act to preserve or stabilize the biological sample or components thereof, and/or may act to extract or concentrate the biological sample or components thereof. In certain embodiments the sample solution may comprise a buffer, optionally containing preservatives, and/or enzymes (protease, nuclease, etc.), and/or surfactants, and/or ATPS components.

In certain embodiments, particularly in point-of-care embodiments, the sample may be applied to the assay device or system described herein immediately or after a modest time interval. In certain embodiments the sample may be delivered to a remote testing facility where the assay is run.

Methods and devices for collecting biological samples are well known to those of skill in the art.

Kits.

In certain embodiments a kit for the detection of a target analyte is provided. In certain embodiments the kit comprises a container containing an ATPS Rehydration and Resolubilization Optimized Wick (ARROW) as described herein. In certain embodiments the ARROW can be provided alone in the container. In certain embodiments the kit can additionally comprise a container containing lateral flow assay (LFA). In certain embodiments the container containing the LFA is a different container than the container containing the ARROW. In certain embodiments a single container contains the ARROW and the LFA. In certain embodiments the ARROW and LFA are assembled together as a joined unit in the single container.

In certain embodiments the kit comprises a container containing a dried nanoconjugate as described herein and a container containing a strip comprising a ATPS components and an LFA strip as described herein.

In certain embodiments the kit comprises instructions (instructional materials) for using the kit for quantification of one or more target analytes.

While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples are offered to illustrate, but not to limit the claimed invention.

### Example 1

### Improved Lateral-Flow Immunoassay for Bacterial and Antibody Biomarkers by Sequential Rehydration of Aqueous Two-Phase Components within a Paper-Based Diagnostic

Our lab developed a method to thermodynamically concentrate target molecules using aqueous two-phase systems (ATPSs) to improve the sensitivity of the lateral-flow immunoassay (LFA) without the need for sample preparation steps. Specifically, we developed the novel concept of sequential ATPS component resolubilization within paper for both PEG/potassium phosphate and UCON-50-HB-5100/potassium phosphate systems and applied it to our diagnostic design, which only required the sample to be added. We visually demonstrated successful ATPS phase separation and further identified the importance of resolubilization order of the dehydrated polymers and salts. Finally, we demonstrated that our novel designs improve the LFA limit of detection for Chlamydia trachomatis bacteria and for human immunoglobulin M (IgM) antibodies by 10-fold, and provide results in less than 15 minutes. This significant advancement in our technology enables the LFA to be operated by untrained or minimally trained personnel, significantly expanding its applicability as a POC test.

Materials and Methods

Preparation of Anti-IgM Antibody-Decorated Gold Nanoparticles (Anti-IgM GNPs)

Citrate-capped gold nanoparticles were synthesized according to Frens and coworkers with slight modifications (Frens (1972) Kolloid-Zeitschrift and Zeitschrift für Polym. 250: 736-741). Briefly, 100 μL of 1% w/v gold(III) chloride hydrate solution was dissolved in 10 mL of UltraPure sterile water (Rockland Immunochemicals Inc., Gilbertsville, Pa.). The solution was stirred and heated to a boil, after which 90 μL of a 2% (w/v) tribasic sodium citrate solution was added. The color of the reaction mixture was allowed to turn red-orange over the course of 10 min. To form functionalized gold nanoprobes (GNPs), 60 μL of 100 mM sodium borate buffer (pH 9) was added to 1 mL of a citrate-capped gold nanoparticle suspension, followed by 16 μg of anti-human IgM antibodies (IgM-Ab). The reaction mixture was placed on a shaker for 30 min to facilitate the formation of dative bonds between the antibodies and the GNPs. 100 μL of 10% w/v bovine serum albumin (BSA) was then added to the suspension and then placed on a shaker for 10 min. Free antibodies were removed by centrifugation and the pellet was resuspended in 100 μL of 100 mM sodium borate buffer (pH 9.0). All materials, chemicals, and reagents were purchased from Sigma-Aldrich (St. Louis, Mo.) unless otherwise specified.

Preparation of Anti-Chlamydia trachomatis Antibody-Decorated Dextran-Coated Gold Nanoparticles (Anti-CT DGNPs)

Dextran-coated gold nanoparticles (DGNPs) were synthesized according to Min and coworkers with slight modifications (Jang et al. (2013) Biomaterials, 34: 3503-3510). Since these DGNPs have been previously shown by our group to provide enhanced stability in high-salt conditions, they were used specifically with the PEG/salt ATPS which requires a higher salt concentration than the UCON/salt system. Briefly, 750 mg of dextran (Mw 15,000-25,000) from Leuconostoc spp. was dissolved in 10 mL of UltraPure sterile water (Rockland Immunochemicals Inc., Gilbertsville, Pa.). The solution was stirred and heated to a boil, after which 135 μL of a 1% w/v gold(III) chloride hydrate solution was added. The color of the reaction mixture turned reddish-violet, and was stirred and boiled for about 20 min. To form functionalized DGNPs, 35 μL of 100 mM sodium borate buffer (pH 9) was added to 1 mL of a DGNP suspension, followed by 16 μg of anti-C. trachomatis antibodies (CT-Ab). The reaction mixture was placed on a shaker for 20 min to facilitate the formation of dative bonds between the antibodies and the DGNPs. 100 μL of 10% w/v BSA was then added to the suspension and then placed on a shaker for 10 min. Free antibodies were removed by centrifugation and the pellet was resuspended in 100 μL of 100 mM sodium borate buffer (pH 9.0).

Preparation of LFA Tests for the Detection of C. trachomatis and IgM

All LFA tests in this study utilized the sandwich assay format. In this format, the presence of the target biomarker in sufficient quantities will produce a red test line, while an absence or insufficient quantity of the biomarker will result in no visible test line. The presence of a control line indicates the completion of flow and the validity of the test. On the LFA tests for C. trachomatis, a solution of 2 mg mL⁻¹ anti C. trachomatis antibodies and 25% w/v sucrose was first printed onto a nitrocellulose membrane to form the test line. Secondary anti-IgG antibodies, which bind to the primary antibodies on the anti-CT DGNPs, were printed downstream of the CT-Ab test line to form the control line. The membrane was then left in a vacuum-sealed desiccation chamber overnight to immobilize the antibodies.

On the LFA tests for human IgM, a solution of 1.5 mg mL⁻¹ anti-human IgM antibodies and 25% w/v sucrose was first printed onto a nitrocellulose membrane to form the test line. A solution of 0.2 mg mL⁻¹ Protein A (Sigma-Aldrich, St. Louis, Mo.), which binds to the primary antibodies on the anti-IgM GNPs, was printed to form the control line. The membrane was also left in a vacuum-sealed desiccation chamber overnight.

Preparation of the ARROW and TUBE Designs

To dehydrate the ATPS and LFA components in paper, pieces of fiberglass paper were cut into appropriate geometries and placed onto a Petri dish. Solutions of the ATPS components were made to the appropriate concentrations and pipetted onto the paper segments. To prepare the ARROW, the ATPS components used were polyethylene glycol (PEG) 8000 and potassium phosphate salt dissolved in phosphate-buffered saline (PBS). To prepare the TUBE design, the ATPS components used were UCON-50-HB-5100 and potassium phosphate salt dissolved in PBS. To dehydrate the components, the paper segments were placed under very low pressure using a Labconco FreeZone 4.5 lyophilizer (Fisher Scientific, Hampton, N.H.) for 2 hours.

Scanning Electron Microscopy (SEM)

Paper segments were cut and treated using the dehydration methods described above. Paper samples included segments dehydrated with 15% (w/w) potassium phosphate, 10% (w/w) PEG, a mixture of 30% (w/w) UCON-50-HB-5100 and 3% (w/w) potassium phosphate, or no additional components (i.e., blank fiberglass). The paper segments were individually placed onto a dry carbon tape-covered holder and sputtered with a metallic coating using a South Bay Technology Ion Beam Sputtering/Etching System (South Bay Technology, San Clemente, Calif.). Samples were imaged at about 500× magnification at 10 kV using a ZEISS Supra 40VP SEM (ZEISS, Irvine, Calif.) at the Electron Imaging Center for NanoMachines and CNSI at UCLA.

Determining the Importance of the Rehydration Order of PEG and Potassium Phosphate

In order to visualize the phase separation of the ATPS on paper, BSA-conjugated DGNPs (BSA-DGNPs), which are burgundy/light purple due to surface plasmon resonance (Daniel & Astruc (2004) Chem. Rev. 104: 293-346; Peter et al. (2007) J. Phys. Chem. 111: 14664-14669), and Brilliant Blue FCF dye (The Kroger Co., Cincinnati, Ohio) were both added to a solution of an ATPS made in PBS. We confirmed that upon completion of the phase separation of this system in a test tube, the BSA-DGNPs partitioned extremely to the PEG-poor phase, while the Brilliant Blue dye partitioned to the PEG-rich phase (Chiu et al. (2014) Lab Chip, 14: 3021-3028). This allowed us to the use the suspension to identify the locations of PEG-poor phase (burgundy/light purple in color), PEG-rich phase (light blue in color), and mixed domain regions (dark blue/dark purple in color) directly on the paper.

Experiments were performed with only a single sheet of the ARROW and without a tapered tip in order to better observe the phase separation behavior. In one condition, the potassium phosphate was dehydrated upstream of the PEG, and in another condition the PEG was dehydrated upstream of the potassium phosphate. In a third condition, the PEG was mixed with potassium phosphate and then dehydrated together. The concentration of the dehydrated components were 15% (w/w) potassium phosphate and 10% (w/w) PEG 8000. Images were taken with a Canon EOS 1000D camera (Canon U.S.A., Inc., Lake Success, N.Y.).

Determining the Rehydration Order of UCON-50-HB-5100 and Potassium Phosphate

In order to first determine the partitioning of the colorimetric indicators in the UCON-50-HB-5100/potassium phosphate ATPS, red-colored BSA-conjugated GNPs (BSA-GNPs) and Brilliant Blue FCF dye were both added to an ATPS solution in PBS. After phase separation in a tube, the BSA-GNPs partitioned extremely to the bottom, UCON-poor phase while the Brilliant Blue dye partitioned into the top, UCON-rich phase. Therefore, for phase separation of dehydrated ATPSs, the location of the UCON-poor phase was identified with the red color of the BSA-GNPs. Similarly, the locations of UCON-rich phase and mixed domain regions were identified with light blue and dark purple color, respectively.

Three different conditions were tested using the UCON/salt system on a single strip of paper. In one condition, the UCON-50-HB-5100 was dehydrated downstream of potassium phosphate, in a second condition the UCON-50-HB-5100 was dehydrated upstream of potassium phosphate, and in the last condition, the UCON-50-HB-5100 was mixed with potassium phosphate and then dehydrated together. The concentrations of the dehydrated components were 30% (w/w) UCON-50-HB-5100 and 3% (w/w) potassium phosphate. Images were taken at different time points with a video camera.

Observing Dynamics of Phase Separation

To visualize phase separation of the dehydrated ATPS systems, we used only the ARROW component of our diagnostic with 15% (w/w) potassium phosphate dehydrated upstream of dehydrated 10% (w/w) PEG 8000. This setup did not contain the LFA membrane or conjugate pad. The suspension containing the BSA-DGNPs and Brilliant Blue dye was allowed to flow along the strip until the fluid reached the end the paper. To visualize phase separation of the dehydrated UCON/salt ATPS, the mixed condition UCON/salt pad was dehydrated with 30% (w/w) and 3% (w/w) potassium phosphate onto a fiberglass paper strip. This setup did not include the LFA membrane or the tube with dehydrated GNPs. A PBS solution containing BSA-GNPs and Brilliant Blue dye was allowed to flow up the strip. Images were captured at different time points using a video camera.

Detection of C. trachomatis Using the Integrated LFA and ARROW

LFA tests were performed to detect varying C. trachomatis concentrations between 0.5 and 500 ng μL⁻¹, such that they were evenly spaced on a logarithmic scale, for the LFA only system and the integrated LFA and ARROW system. The sample suspensions contained C. trachomatis (EastCoast Bio, North Berwick, Me.) diluted in PBS. The sample solution volumes were 70 and 600 μL per test for the control and dehydrated ATPS conditions, respectively. A smaller sample volume was used for the control because it did not have the ARROW component, and therefore, did not require as much sample volume to run the test. The control LFA strip was comprised of a sample pad (treated with 1% BSA), a conjugate pad containing the anti-CT DGNPs, a nitrocellulose membrane, and an absorbent pad. The integrated design substituted the initial sample pad with the ARROW component. We did not include a blank paper wick to mimic the ARROW component in the control since comparing to the case without the wick was a more stringent comparison as C. trachomatis can be lost in a blank wick. The tests were allowed to run for 15 minutes before images were taken with a Canon EOS 1000D camera.

Detection of Human IgM Using the Integrated LFA and TUBE

LFA tests were conducted on sample solutions of human IgM (EastCoast Bio, North Berwick, Me.) in PBS, with varying human IgM concentrations from 0.01 to 10 ng μL⁻¹. Here, the sample volumes used for the control case and the dehydrated ATPS conditions were 25 μL and 150 μL, respectively. The control LFA strip was comprised of a sample pad (treated with 1% BSA), a conjugate pad containing the anti-IgM GNPs, a nitrocellulose membrane, and an absorbent pad. In the TUBE design, the sample pad and conjugate pad were omitted and replaced with the dehydrated UCON/salt strip and a spacer pad treated with 1% BSA in water. GNPs of an equal amount as the control case were mixed with BSA to a total BSA concentration of 1% (w/v), and applied to a microcentrifuge tube. The tube was then placed under very low pressure using a Labconco FreeZone 4.5 lyophilizer (Fisher Scientific, Hampton, N.H.) for 1 hour, leaving the GNPs in dried powder form.

To run the test using the TUBE design, an IgM sample was added to the sample tube to rehydrate the GNPs and allow binding to the target. Then, the test strip with the dehydrated UCON/salt pad was dipped into the tube and the sample was allowed to wick up the strip toward the absorbent pad. The tests were allowed to run for 12 minutes before images were taken with a Canon EOS 1000D camera.

Quantitative Image Analysis

Images were analyzed using a customized MATLAB script previously developed and described by our lab (Jue et al. (2014) Biotechnol. Bioeng. 111: 2499-2507). Briefly, in this program, LFA images are cropped just inside the edges of the membrane before being analyzed. The program takes several calibration images of a positive test with visible control and test lines, and uses those to determine the length from the control line to the test line. It then analyzes the experimental images by determining the average pixel intensity on the test line and subtracting the average pixel intensity of the membrane background. Finally, it returns the relative test line signal as a percentage of the maximum signal intensity tested (which is produced by the highest concentration tested). The pixel intensity was plotted using GraphPad Prism.

Results and Discussion

Importance of the Rehydration Order of PEG and Potassium Phosphate

Our novel ARROW design introduces the unexplored concept of phase separation after sequential ATPS component resolubilization during fluid flow, which is in contrast to the traditional method of ATPS research that examines phase separation in a stagnant solution with an initial homogenous distribution of ATPS components. Therefore, we investigated the effect of the PEG and potassium phosphate rehydration order on the phase separation behavior within the paper. To do this, we utilized a suspension comprised of BSA-DGNPs and Brilliant Blue dye which allowed us to visualize the phase separation process as the suspension flowed through the paper, a technique previously utilized by our laboratory (Chiu et al. (2014) Lab Chip, 14: 3021-3028). In short, the BSA-DGNPs partitioned into the PEG-poor phase indicated by the burgundy/light purple color, while the blue dye partitioned into the PEG-rich phase indicated by the light blue color. Regions of macroscopically mixed domains contained both BSA-DGNPs and blue dye, indicated by the dark blue/dark purple color. During fiberglass paper preparation, we altered the location of the dehydrated ATPS components, such that one condition had the dehydrated potassium phosphate located upstream of the dehydrated PEG (denoted ‘Salt→PEG’), one condition had the dehydrated PEG located upstream of the dehydrated potassium phosphate (denoted ‘PEG→Salt’), and a third condition contained a mixture of the PEG and potassium phosphate dehydrated across the entire length of the strip.

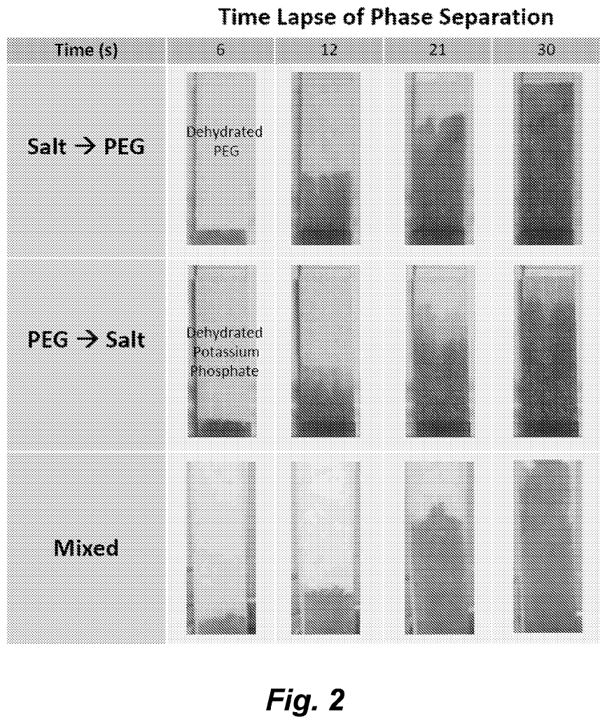

From these results (FIG. 2), we note several interesting observations. First, the ‘mixed’ condition resulted in no visible phase separation as the entire strip appeared purple due to the mixture of PEG-rich and PEG-poor domains. In addition, the leading PEG-poor fluid had a significantly darker burgundy color in the ‘Salt→PEG’ condition compared to the ‘PEG→Salt’ condition, indicating that the ‘Salt→PEG’ condition contained more BSA-DGNPs in the leading fluid, and therefore, is more effective at concentrating large species. Furthermore, the PEG-rich phase exhibited significantly more volumetric growth over time in the ‘Salt→PEG’ condition compared to the PEG-rich phase in the ‘PEG→Salt’ condition. This suggests that, in the ‘Salt→PEG’ condition, the newly formed PEG-poor domains are able to get out of the mixed domain region and more efficiently pass through the trailing PEG-rich phase and collect into the leading PEG-poor phase. This results in the PEG-rich phase becoming larger as the mixed domains region becomes smaller. One possible reason for this phenomenon is the formation of PEG-poor channels within the PEG-rich phase that connect to the leading PEG-poor phase. Research in multiphase fluid flow within porous media has found that less viscous fluids will develop preferred channels when displacing more viscous fluids (Wooding & Morel-Seytoux (1976) Annu. Rev. Fluid Mech. 8: 233-274).

We hypothesized that switching the location of the ATPS components, such that PEG is resolubilized prior to potassium phosphate, reduces or prevents the formation of PEG-poor channels. When considering a sample solution flowing through the ‘PEG→Salt’ condition at the location that the leading fluid transitions from the dehydrated PEG region to the dehydrated potassium phosphate region, the fluid contains a high concentration of resolubilized PEG and no potassium phosphate. As the fluid flows into the dehydrated potassium phosphate region, the concentration of potassium phosphate increases and phase separation occurs. If this situation is examined from the perspective of a traditional PEG and potassium phosphate phase diagram (Hatti-kaul (2000) Aqueous Two-Phase Systems Methods and Protocols, 1st ed. Humana Press), initial phase separation in this leading fluid will occur at the region of high PEG and low potassium phosphate concentrations. This initial phase separation would result in a large PEG-rich phase volume and a small PEG-poor phase volume, as described by the lever rule (Hatti-kaul (2000) Aqueous Two Phase Systems Methods and Protocols, 1st ed. Humana Press; Morse (1997) 1 Geol. 105: 471-482). We hypothesized that the larger volume of the initial PEG-rich phase prevents PEG-poor channels from being formed and connecting to the leading PEG-poor phase. This would hinder subsequently formed PEG-poor domains from passing through and collecting into the leading fluid. This hypothesis is supported by our observations of the ‘PEG→Salt’ condition, notably: (i) the lower concentration of BSA-DGNPs in the leading PEG-poor phase, indicated by the lighter burgundy color, and (ii) the presence of a macroscopically mixed domain region, located behind the PEG-rich phase, indicated by the dark purple color. From these observations, we decided to use the ‘Salt→PEG’ condition in the final design incorporated with the LFA.

Importance of the Rehydration Order of UCON-50-HB-5100 and Potassium Phosphate

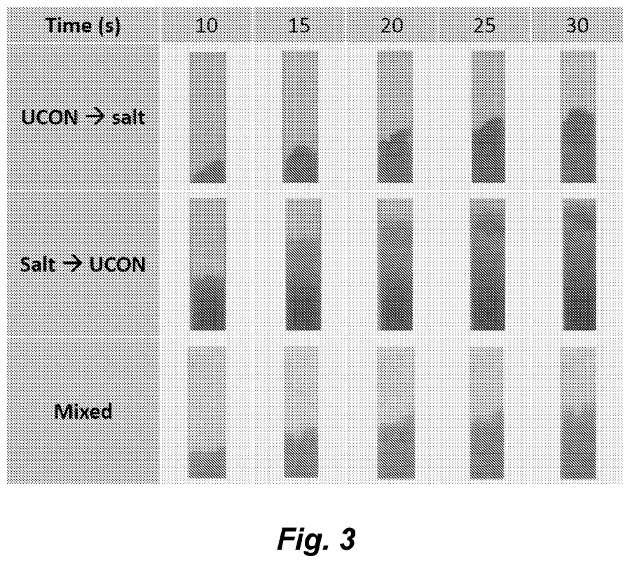

In the TUBE design, we investigated the order of UCON-50-HB-5100 and potassium phosphate rehydration order on the phase separation behavior in paper, using the same colorimetric indicators as previously described. Three different combinations were tested (FIG. 3): one in which the dehydrated potassium phosphate was located upstream of the dehydrated UCON-50-11B-5100 (‘Salt→UCON’), one in which the dehydrated UCON-50-HB-5100 was located upstream of the dehydrated potassium phosphate (‘UCON→Salt’), and one in which the two components were mixed together and applied evenly along the entire fiberglass strip (‘Mixed’). We observed that the ‘UCON→Salt’ condition resulted in very little noticeable separation, as can be seen by the purple color caused by the blending of both the mixed domains of BSA-GNPs and the blue dye along the strip. This is in agreement with the hypothesis that a high volume of a highly concentrated UCON-rich phase prevents the formation of UCON-poor channels, and in this case, completely prevents the formation of a distinct UCON-poor leading front. On the other hand, phase separation was observed in the ‘Salt→UCON’ condition, in which the leading front containing the GNPs was visible within 15 seconds. In the ‘mixed’ condition, we noticed phase separation occurring within 10 seconds, indicating that rehydrating a mixture of UCON and potassium phosphate does not hinder the collection of UCON-poor domains and the formation of the UCON-poor phase. Although the ‘mixed’ condition produced a leading front volume approximately equal to that of the ‘Salt→UCON’ case, it also produced a lower flow rate, which has been shown to provide additional benefits in improving the LFA detection limit (Choi et al. (2016) Anal. Chem. 88: 6254-6264). For this reason, the ‘mixed’ condition was used in the design later incorporated with the LFA.

Dynamics of Phase Separation

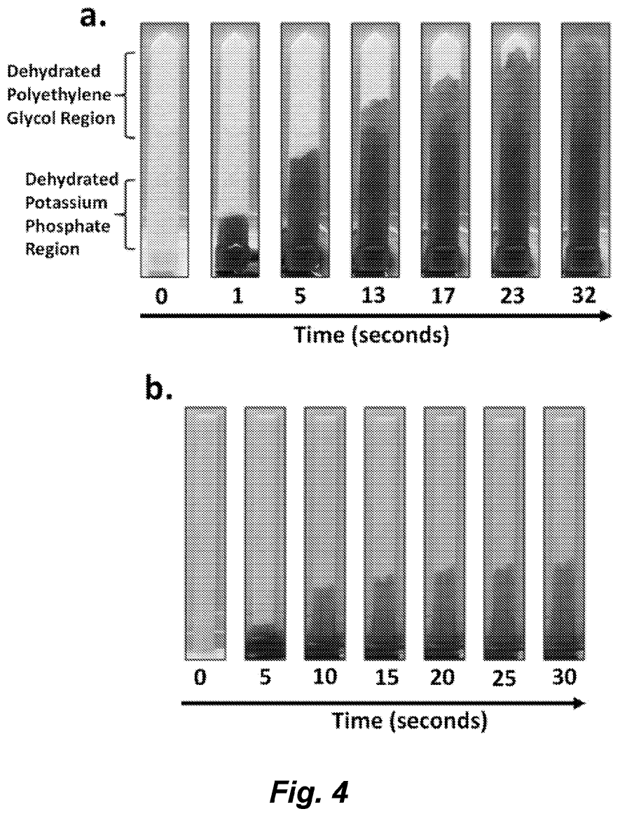

Once the rehydration conditions for the two ATPSs were optimized we then made more detailed observations of the phase separation time within these two systems. It was important to demonstrate that our methods of dehydration allowed for rapid rehydration of the ATPS components during the flow of the sample solution through the diagnostic. As shown in FIG. 4, panel a, we observed successful phase separation using our ARROW setup, in which phase separation occurred shortly after the suspension flowed into the dehydrated PEG region. We also noticed that the PEG-poor region collected into the leading fluid in front of the PEG-rich region, mimicking an important phenomenon discovered in our previous work (Chiu et al. (2014) Lab Chip, 14: 3021-3028), which is necessary considering that the PEG-poor region will contain the concentrated C. trachomatis and needs to be in the leading fluid when flowing through the conjugate pad. The process of flowing through the ARROW only took approximately 30 s.

Interestingly, we observed that the PEG-poor region in the leading fluid expanded as the fluid flowed through the dehydrated PEG region, which is best observed in the transition from time points 13 s to 23 s. During this time period we also observed that the PEG-rich region expanded but maintained its initial location at the beginning of the dehydrated PEG region. These two observations together suggest that the dehydrated PEG and potassium phosphate quantities are sufficient to continue phase separation after initial phase separation in the leading fluid, and that the newly formed PEG-poor domains are flowing through the PEG-rich region to collect at the leading PEG-poor region.

Phase separation was also seen in the mixed UCON-salt design (FIG. 4, panel b) within 10 s. Here, the UCON-poor region containing BSA-GNPs collected into the leading fluid front, concentrating the GNPs from the large initial solution into a small volume, which remained consistent throughout the duration of the flow study. After the phase separation (10 s to 30 s), there was a noticeable decrease in the flow speed through the strip, which is likely attributed to the formation of the viscous UCON-rich lagging phase.

Integrating the LFA with the Dehydrated ATPS

We then used dehydrated components of the PEG/salt ATPS and the UCON/salt ATPS to produce two different assay designs. Our dehydrated PEG/salt ATPS diagnostic device (FIG. 5) was comprised of two major components: the ARROW and the standard LFA. The ARROW consisted of several fiberglass paper sheets layered together. Considering that the function of the ATPS is to concentrate the target pathogen, it was necessary that the ARROW was able to wick up a large volume of sample solution. 15% (w/w) potassium phosphate was dehydrated in the upstream portion of each fiberglass sheet, while 10% (w/w) PEG 8000 was dehydrated in the downstream portion of each fiberglass sheet. It was important to leave blank space between the dehydrated PEG and the tip of the sheet to allow for collection of the PEG-poor phase that contained the concentrated pathogen. The downstream tip of each sheet was tapered to form a point, which facilitates proper transition of the liquid into the conjugate pad.

The LFA portion of the diagnostic consisted of the conjugate pad, containing the colorimetric indicator, connected to a nitrocellulose membrane with printed primary and secondary antibodies, and followed by an absorbent pad. The LFA portion interfaced with the ARROW by fitting a small upstream portion of the conjugate pad perpendicularly into a slit that had been cut in the ARROW.

The ARROW was designed to concentrate a biomarker capable of partitioning to a single phase on its own. Since the C. trachomatis whole bacteria is relatively large (0.8 to 1 μm), it can partition extremely to the PEG-poor phase without intervention. However, many infectious disease biomarker targets, such as the HIV antibodies typically detected in HIV rapid tests, are smaller in scale and do not partition extremely to a single phase. Therefore, another strategy must be utilized to concentrate these biomarkers. Previously, our group demonstrated that the gold nanoparticle conjugates typically used in LFA can be added directly into an ATPS, where they partition extremely to the polymer-poor phase in a polymer/salt ATPS (Mashayekhi et al. (2012) Anal. Bioanal. Chem. 404: 2057-2066; Chiu et al. (2014) Ann. Biomed. Eng. 42(11): 2322-2332). In this format, the GNPs were added to the UCON/salt sample solution and were allowed to bind the target present in solution before phase separation occurred. After the onset of phase separation, the large GNP-target complexes partitioned to the UCON-poor phase, thus concentrating the target into the UCON-poor phase. Extraction of the GNPs and application to the LFA yielded improvements in the detection limit of these protein targets. In this study, we focused on incorporating this mechanism into the dehydrated format to concentrate smaller targets, using a human IgM antibody (970 kDa, or approximately 37 nm in diameter) as a model biomarker target.

The TUBE design (FIG. 6) is comprised of two main components: the sample tube, and the test strip that consists of the UCON/salt pads connected to the standard LFA. In this design, it is imperative that the GNPs access the entire sample solution and bind to the target prior to the ATPS concentration step. It is also important that after binding the target, the GNPs access the dehydrated ATPS region at the same time in order to maximize the GNPs that become concentrated into the resulting UCON-poor leading front. One approach to achieve these design criteria was to dry the conjugates and store them in powder form housed in a sample microcentrifuge tube. In this case, the liquid sample is first added into the tube, in which the GNPs are resolubilized and immediately bind to any human IgM present. Next, the test strip is added into the sample tube, and the GNPs collectively wick up the test strip, first making contact with the UCON/salt pad. When this occurs, the dehydrated UCON/salt mixture is rehydrated by the wicking solution, inducing the formation and separation of the UCON-rich and the UCON-poor phases. The GNPs are concentrated in the newly-formed UCON-poor fluid front, while the newly-formed and more viscous UCON-rich region lags behind. The spacer pad contains BSA to ensure even transition of the UCON-poor phase into the nitrocellulose-based detection region and prevent nonspecific binding of the GNPs.

The SEM image (FIG. 5) of the blank fiberglass region of the fiberglass paper shows a porous fiber-based matrix structure. The dehydrated PEG, potassium phosphate, and mixed UCON-50-HB-5100/potassium phosphate regions show a similar porous structure, with the addition of web-like connections, which we believe contain a majority of their respective ATPS components (FIGS. 5 and 6). These images demonstrate that the process of dehydration does not significantly deform the porous structure of the fiberglass paper, which is critical for proper wicking of the sample fluid. An SEM image of the nitrocellulose paper (FIG. 6) shows a typical pore structure and size that accommodates transport of the sample fluid.

Improved Limit of Detection for C. trachomatis and Human IgM Using the Integrated LFA and Dehydrated ATPS

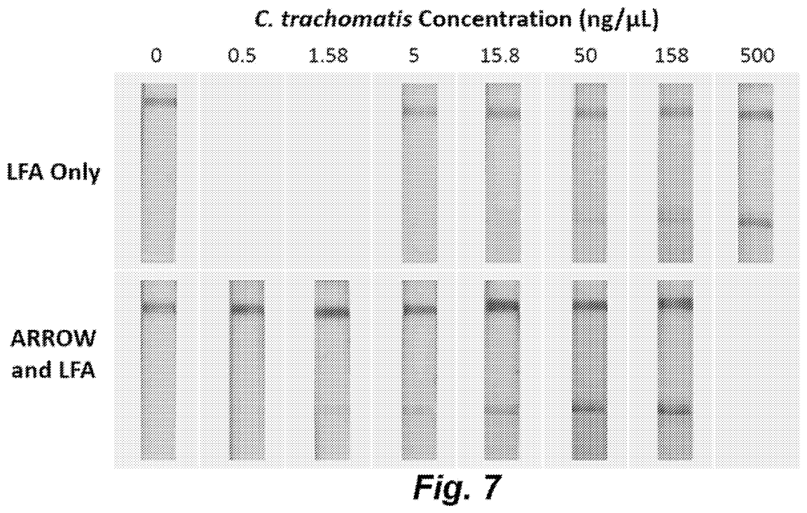

We then demonstrated that our ARROW design effectively concentrated a C. trachomatis sample suspension, resulting in an improved limit of detection for LFA. To do this, we ran sample suspensions of varying initial concentrations of C. trachomatis on LFA test strips, with and without the ARROW component. We see from the results of the LFA panel (FIG. 7) that the LFA only system started showing false negative results at around 15.8 ng μL⁻¹ C. trachomatis while the integrated LFA and ARROW system started showing false negative results at around 1.58 ng μL⁻¹ C. trachomatis. This visually demonstrates a 10-fold improvement in the limit of detection.

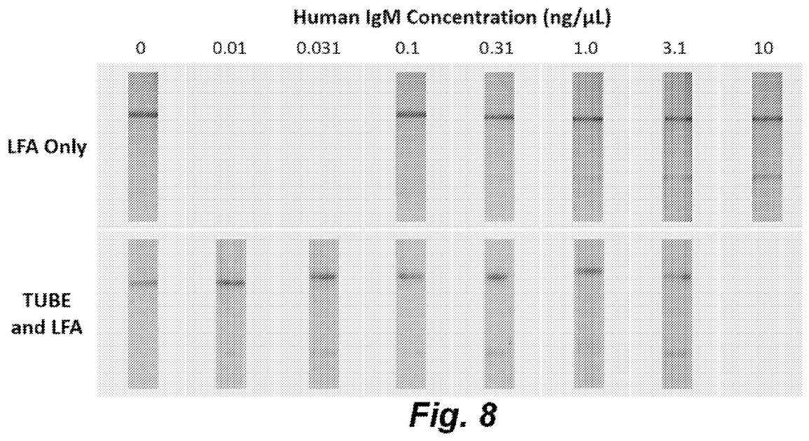

Lastly, we demonstrated that we could use the TUBE diagnostic to effectively concentrate human IgM in a PBS sample and improve the LFA limit of detection (FIG. 8). In this case, the detection limit of the LFA control was determined to be 0.31 ng μL⁻¹. On the other hand, the integrated TUBE and LFA system was able to accurately detect human IgM at 0.031 ng μL⁻¹, visually demonstrating a 10-fold improvement in the limit of detection compared to the LFA control.

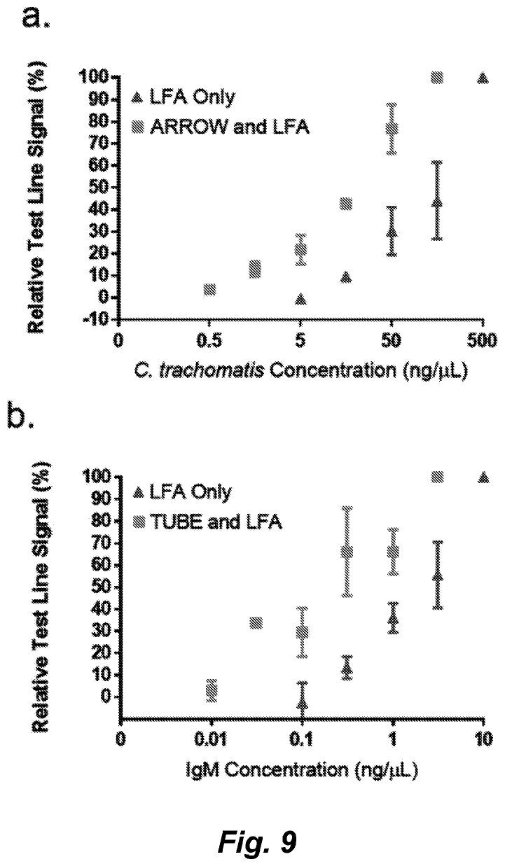

We also quantified the pixel contrast of the test lines on the LFA images using a customized MATLAB program developed and described by our laboratory (FIG. 9) (Jue et al. (2014) Biotechnol. Bioeng. 111: 2499-2507). This allowed us to quantitatively assess the improvement in the limit of detection. For any given concentration of C. trachomatis, we see a significant increase in the relative test line intensity for the integrated ARROW and LFA system compared to the LFA only system. For example, at 50 ng C. trachomatis, the LFA only condition had a relative intensity of 30.3%±10.8%, while the integrated ARROW and LFA had a relative intensity of 76.8%±11.1%. Similar results were seen in the image analysis of the IgM tests with the integrated TUBE and LFA at all IgM concentrations. For example, at 1.0 ng μL⁻¹ IgM, the LFA only condition had a relative pixel intensity of 36.1%±6.6%, while the integrated TUBE and LFA had a pixel contrast intensity of 66.1%±10.0%. In both cases, the image analysis was able to detect test lines with significantly greater intensities than the background at lower concentrations when the dehydrated ATPS components were integrated.

### CONCLUSIONS

In the current study, we present two new paper-based diagnostic designs that are capable of thermodynamic target concentration through dehydration of ATPS components. With these paper-based devices, only the sample needs to be added without additional sample preparation steps. We used the dehydrated PEG/potassium phosphate salt ATPS within the ARROW design to concentrate and detect C. trachomatis, and used the dehydrated UCON-50-HB-5100/potassium phosphate ATPS within the TUBE design to concentrate and detect human IgM. Specifically, we demonstrated that the ARROW and the TUBE designs improved the LFA limit of detection for their respective biomarker targets by 10-fold, while still providing results in less than 15 minutes.

An LFA diagnostic with improved sensitivity, that still maintains its low cost, rapid time to result, and ease of use, will significantly increase its applicability as a POC screening test for infectious diseases. We have demonstrated that the dehydrated ATPS technology can be applied to a variety of different targets suitable for detection by LFA. Most LFA-based diagnostics for infectious diseases are not developed or not used due to poor sensitivity. Considering that the dehydrated ATPS can improve LFA sensitivity without adding any additional steps to the user, our novel technology has the potential to create many viable infectious disease LFA tests, both for use by physicians and as over-the-counter tests.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A wick for concentrating an analyte within an aqueous two-phase extraction system in a paper, said wick comprising:
a paper configured to receive a sample wherein said paper comprises:
a first region containing a first component of an aqueous two-phase system (ATPS) where said first component is in a dry form; and
a second region containing a second component of an aqueous two-phase system (ATPS) where said second component is in a dry form;
wherein said first region and said second region are disposed so that when said wick is contacted with a fluid sample, said first component of said ATPS is hydrated before said second component; or
wherein said paper comprises a region containing both a first component of an aqueous two-phase system (ATPS) and a second component of an aqueous two-phase system where said first component and said second component are in a dry form so that when said wick is contacted with a fluid sample, said first component of said ATPS and said second component of said ATPS are hydrated at substantially the same time.

2. The wick of claim 1, wherein said paper comprises:
a first region containing a first component of an aqueous two-phase system (ATPS) where said first component is in a dry form; and
a second region containing a second component of an aqueous two-phase system (ATPS) where said second component is in a dry form;
wherein said first region and said second region are disposed so that when said wick is contacted with a fluid sample, said first component of said ATPS is hydrated before said second component.

3. The wick according to any one of claims 1-2, wherein said wick is configured so that the first component of said ATPS when hydrated flows into said second component of said ATPS hydrating said second component to provide a mixed phase that separates into a first phase comprising said first component and a second phase comprising said second component as the ATPS moves through said wick.

4. The wick according to any one of claims 1-3, wherein said first component and said second component are components of a polymer/salt ATPS where said first component comprises a salt and said second component comprises a polymer.

5. The wick of claim 4, wherein said salt comprise one or more salts selected from the group consisting of potassium phosphate, sodium sulfate, magnesium sulfate, ammonium sulfate, sodium citrate, magnesium chloride, magnesium citrate, magnesium phosphate, sodium chloride, potassium citrate, and potassium carbonate.

6. The wick of claim 5, wherein said salt comprises potassium phosphate.

7. The wick according to any one of claims 4-6, wherein said salt ranges from about 0.1% w/w to about 40% w/w, or from about 1% w/w up to about 30% w/w, or from about 5% w/w up to about 25% w/w, or from about 10% w/w up to about 20% w/w.

8. The wick of claim 7, wherein said salt is present at about 15% (w/w).

9. The wick according to any one of claims 4-8, wherein said polymer comprises a polymer selected from the group consisting of polyethylene glycol (PEG), ethylene/propylene copolymer (e.g., UCON™ 50-HB), propylene glycol (PPG), methoxypolyethylene glycol, and polyvinyl pyrrolidone.

10. The wick of claim 9, wherein said polymer comprises polyethylene glycol (PEG).

11. The wick of claim 10, wherein said PEG has a molecular weight that ranges from about 1,000 to about 100,000, or from about 4,000 to about 50,000, or from about 5,000 up to about 40,000, or up to about 30,000, or up to about 20,000.

12. The wick of claim 11, wherein said polymer comprises polyethylene glycol (PEG) 8000 MW.

13. The wick according to any one of claims 4-12, wherein said polymer comprises about 1% w/w to about 30% w/w, or from about 5% w/w up to about 25% w/w, or from about 10% w/w up to about 25% w/w, or from about 10% w/w up to about 20% w/w polymer.

14. The wick of claim 13, wherein said polymer comprises about 10% (w/w).

15. The wick according to any one of claims 1-14, wherein said paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), and combinations thereof.

16. The wick of claim 15, wherein said paper comprises fiberglass.

17. The wick according to any one of claims 1-16, wherein said wick comprises a plurality of layers of said paper.

18. The wick of claim 17, wherein said wick comprises at least 3, or at least 4, or at least 5, or at least 6, or at least 7, or at least 8, or at least 9, or at least 10, or at least 15, or at least 20 layers of said paper.

19. The wick of claim 17, wherein said wick comprises about 5 layers of said paper.

20. The wick according to any one of claims 1-19, wherein an ATPS component free region is disposed between said first region and said second region.

21. The wick according to any one of claims 1-19, wherein said first region is disposed adjacent to said second region.

22. The wick according to any one of claims 1-21, wherein said wick comprises a sample application region.

23. The wick of claim 22, wherein said sample application region comprises a sample pad.

24. The wick according to any one of claims 1-23, wherein said wick tapers in a region downstream from said second region and upstream of a lateral flow assay (LFA) when an LFA is in fluid communication with said wick.

25. The wick according to any one of claims 1-24, wherein said wick is configured to be coupled to a lateral flow immunoassay (LFA) and provide fluid communication from said wick to said LFA.

26. The wick of claim 25, wherein said wick is configured to be coupled to an LFA so that plane of wick is perpendicular to the plane of the LFA.

27. The wick of claim 25, wherein said wick is configured to be coupled to an LFA so that plane of wick is parallel to the plane of the LFA.

28. The wick of claim 25, wherein said wick is coupled to a lateral flow immunoassay.

29. The wick of claim 28, wherein said wick is coupled to an LFA so that plane of said wick is parallel to the plane of the LFA.

30. The wick of claim 28, wherein said wick is coupled to an LFA so that plane of said wick is perpendicular to the plane of the LFA.

31. The wick according to any one of claims 28-30, wherein said lateral flow assay comprises:
an LFA paper comprising:
a conjugate region containing a conjugate comprising an indicator moiety attached to a binding moiety that binds to the analyte to be detected, or configured to receive a nanoconjugate complexed with said analyte;
an absorbent region; and
a detection zone comprising a moiety that captures an analyte/nanoconjugate complex.

32. The wick of claim 31, wherein said detection zone comprise a detection line.

33. The wick according to any one of claims 31-32, wherein said LFA comprises a control zone comprising a moiety that captures an analyte/nanoconjugate complex and said nanoconjugate absent said analyte.

34. The wick according to any one of claims 31-33, wherein said control zone comprises a control line.

35. The wick according to any one of claims 31-34, wherein said conjugate region comprises a conjugate pad.

36. The wick according to any one of claims 31-35, wherein said absorbent region comprises an absorbent pad.

37. The wick according to any one of claims 31-36, wherein said LFA paper is the same material as the paper comprising said wick.

38. The wick according to any one of claims 31-37, wherein said LFA paper is a different material than the paper comprising said wick.

39. The wick according to any one of claims 31-38, wherein said LFA paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

40. The wick of claim 39, wherein said LFA paper comprises nitrocellulose.

41. The wick of claim 39, wherein said LFA paper comprises fiberglass.

42. The wick according to any one of claim 22-23 or 31-41, wherein the sample application region of said wick or the conjugate region of said LFA contains a nanoconjugate comprising an indicator moiety attached to an analyte binding moiety that binds to the analyte to be detected.

43. The wick of claim 42, wherein said analyte binding moiety is selected from the group consisting of an antibody, a lectin, a protein, a glycoprotein, a nucleic acid, monomeric nucleic acid, a polymeric nucleic acid, an aptamer, an aptazyme, a small molecule, a polymer, a lectin, a carbohydrate, a polysaccharide, a sugar, and a lipid.

44. The wick of claim 43, wherein said analyte binding moiety comprises an antibody that binds to said analyte.

45. The wick according to any one of claims 42-44, wherein said indicator comprises a moiety selected from the group consisting of a colorimetric indicator, a fluorescent indicator, and a moiety that can be bound by a construct comprising a colorimetric or fluorescent indicator.

46. The wick according to any one of claims 42-45, wherein said indicator comprise a material selected from the group consisting of a synthetic polymer, a metal, a mineral, a glass, a quartz, a ceramic, a biological polymer, a plastic, and combinations thereof.

47. The wick according to any one of claims 42-46, wherein said indicator comprises a colorimetric indicator.

48. The wick of claim 47, wherein said indicator comprises a gold nanoparticle.

49. A system for the detection of an analyte, said system comprising:
a container containing a dried nanoconjugate comprising an indicator moiety attached to an analyte binding moiety that binds to said analyte; and
a device comprising a first paper containing components of an aqueous two-phase system where said first paper is in fluid communication with a lateral flow assay (LFA), and where said first paper comprises:
a first region containing a first component of an aqueous two-phase system (ATPS) where said first component is in a dry form; and
a second region containing a second component of an aqueous two-phase system (ATPS) where said second component is in a dry form;
wherein:
said first region and said second region are disposed so that when said wick is contacted with a fluid sample, said first component of said ATPS is hydrated before said second component; or
said first region and said second region are the same region and said first component and second component are each distributed over substantially the same region.

50. The system of claim 49, wherein said first region and said second region are the same region and said first component and second component are each distributed over substantially the same region.

51. The system according to any one of claims 49-50, wherein said first component and said second component are components of a polymer/salt ATPS where said first component comprises a salt and said second component comprises a polymer.

52. The system of claim 51, wherein said salt comprise one or more salts selected from the group consisting of potassium phosphate, sodium sulfate, magnesium sulfate, ammonium sulfate, sodium citrate, magnesium chloride, magnesium citrate, magnesium phosphate, sodium chloride, potassium citrate, and potassium carbonate.

53. The system of claim 52, wherein said salt comprises potassium phosphate.

54. The system according to any one of claims 51-53, wherein said polymer comprises a polymer selected from the group consisting of polyethylene glycol (PEG), ethylene/propylene copolymer (e.g., UCON™ 50-HB), propylene glycol (PPG), methoxypolyethylene glycol, and polyvinyl pyrrolidone.

55. The system of claim 54, wherein said polymer comprises ethylene/propylene copolymer (e.g., UCON™ 50-BB).

56. The system according to any one of claims 49-55, wherein said first paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

57. The system of claim 56, wherein said first paper comprises fiberglass.

58. The system according to any one of claims 49-57, wherein said first paper comprises a single layer of said paper.

59. The system according to any one of claims 49-57, wherein said first paper comprises a plurality of layers of said paper.

60. The system of claim 59, wherein said first paper comprises at least 3, or at least 4, or at least 5, or at least 6, or at least 7, or at least 8, or at least 9, or at least 10, or at least 15, or at least 20 layers of said paper.

61. The system according to any one of claims 49-60, wherein a spacer is disposed between said first paper and said lateral flow assay where said spacer provides fluid communication between said first paper and said lateral flow assay.

62. The system of claim 61, wherein said spacer is treated to reduce non-specific binding of analyte and/or nanoconjugate and/or nanoconjugate/analyte complex.

63. The system of claim 62, wherein said spacer is treated with BSA.

64. The system according to any one of claims 62-63, wherein said spacer comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

65. The system of claim 64, wherein said spacer paper comprises fiberglass.

66. The system according to any one of claims 49-60, wherein said paper is disposed adjacent to lateral flow assay.

67. The system according to any one of claims 49-66, wherein said lateral flow assay comprises:
an LFA paper comprising:
an absorbent region; and
a detection zone comprising a moiety that captures an analyte/nanoconjugate complex.

68. The system of claim 67, wherein said detection zone comprises a detection line.

69. The system according to any one of claims 67-68, wherein said LFA comprises a control zone comprising a moiety that captures an analyte/nanoconjugate complex and said nanoconjugate absent the presence of said analyte.

70. The system of claim 69, wherein said control zone comprises a control line.

71. The system according to any one of claims 67-70, wherein said absorbent region comprises an absorbent pad.

72. The system according to any one of claims 67-71, wherein said LFA paper is the same material as said first paper.

73. The system according to any one of claims 67-71, wherein said LFA paper is a different material than said first paper.

74. The system according to any one of claims 67-73, wherein said LFA paper comprises a material selected from the group consisting of a cellulose, a fiberglass, a nitrocellulose, a polyvinylidene fluoride, a nylon, a charge modified nylon, a polyethersulfone, a polytetrafluoroethylene (PTFE), a polyester, and combinations thereof.

75. The system of claim 74, wherein said LFA paper comprises nitrocellulose.

76. The system according to any one of claims 49-75, wherein analyte binding moiety is selected from the group consisting of an antibody, a lectin, a protein, a glycoprotein, a nucleic acid, monomeric nucleic acid, a polymeric nucleic acid, an aptamer, an aptazyme, a small molecule, a polymer, a lectin, a carbohydrate, a polysaccharide, a sugar, and a lipid.

77. The system of claim 76, wherein said analyte binding moiety comprises an antibody that binds to said analyte.

78. The system according to any one of claims 76-77, wherein said indicator comprises a moiety selected from the group consisting of a colorimetric indicator, a fluorescent indicator, and a moiety that can be bound by a construct comprising a colorimetric or fluorescent indicator.

79. The system according to any one of claims 76-78, wherein said indicator comprise a material selected from the group consisting of a synthetic polymer, a metal, a mineral, a glass, a quartz, a ceramic, a biological polymer, a plastic, and combinations thereof.

80. The system according to any one of claims 76-79, wherein said indicator comprises a colorimetric indicator.

81. The system of claim 80, wherein said indicator comprises a gold nanoparticle.

82. A method of detecting and/or quantifying an analyte in a sample, said method comprising:
providing an aqueous solution or suspension comprising said sample; and
applying said solution to a wick according to any one of claims 1-48 where said solution sequentially hydrates said first component and said second component as said solution migrates through said wick and partitions said analyte into a phase of said ATPS;
delivering said ATPS into said lateral flow assay; and
detecting and/or quantifying said analyte in said lateral flow assay if said analyte is present.

83. The method of claim 82, wherein said delivering comprises contacting a wick according to any one of claims 1-30 with a sample receiving region of said lateral flow assay.

84. The method of claim 82, wherein said wick is in fluid communication with a said wick and said ATPS flows into said LFA.

85. The method of claim 84, wherein said wick is a wick according to any one of claims 28-48.

86. A method of detecting and/or quantifying an analyte in a sample, said method comprising:
providing a system according to any one of claims 49-81;
introducing said sample into said container containing a dried nanoconjugate to hydrate said nanoconjugate and to contact said nanoconjugate with said sample where said nanoconjugate forms a nanoconjugate/analyte complex when said analyte is present in said sample;
contacting the region of said device comprising said components of an aqueous two-phase system and hydrating said components where said hydrated components flow through said lateral flow assay; and
detecting and/or quantifying said analyte in said lateral flow assay if said analyte is present.

87. The method according to any one of claims 82-86, wherein said sample is not processed prior to application to said device.

88. The method according to any one of claims 82-86, wherein said sample is diluted prior to application to said device.

89. The method of claim 88, wherein said sample is diluted with phosphate-buffered saline (PBS).

90. The method according to any one of claims 82-89, wherein said subject is a human.

91. The method according to any one of claims 82-89, wherein said subject is a non-human mammal.

92. The method according to any one of claims 82-91, wherein said sample is selected from the group consisting of a biological sample (e.g., oral fluid or tissue sample, nasal fluid, urine, blood or blood fraction, cerebrospinal fluid, lymph, tissue biopsies, vaginal samples, and the like), a food sample, and an environmental sample.

93. The method according to any one of claims 82-92, wherein said analyte comprises a bacterium, a fungus, a protozoan, a virus, or a component thereof.

94. The method according to any one of claims 82-92, wherein said analyte comprises a marker of an infection.

95. The method of claim 94, wherein said marker comprises an antibody directed against the infecting pathogen (e.g., an anti-HIV antibody).

96. A kit comprising:
a container containing a wick according to any one of claims 1-48; and/or
a container containing the container and/or the device of the system according to any one of claims 49-82.
